(19) United States
(12) Patent Application Publication
(10) Pub. No.: US 2021/0236126 A1
(43) Pub. Date: Aug. 5, 2021

(54) TISSUE CARE DEVICE AND METHOD OF USE

(71) Applicants: Maruho Medical, Inc., Reno, NV (US); Innovative Drive Corporation, Reno, NV (US)

(72) Inventors: Matthew Byrnes NEWELL, Reno, NV (US); Luke W. CLAUSON, Reno, NV (US); Akihiko IKOMA, Osaka (JP)

(73) Assignees: Maruho Medical, Inc., Reno, NV (US); Innovative Drive Corporation, Reno, NV (US)

(21) Appl. No.: 17/201,821

(22) Filed: Mar. 15, 2021

Related U.S. Application Data
(63) Continuation of application No. PCT/US2019/048347, filed on Aug. 27, 2019.
(60) Provisional application No. 62/732,979, filed on Sep. 18, 2018.

(51) Int. Cl. A61B 17/08

(52) U.S. Cl. CPC A61B 17/08; A61B 2017/081; A61B 17/085

(57) ABSTRACT

Tissue care devices and methods of using the same are disclosed. The tissue care devices can have one or multiple expanded configurations and one or multiple contracted configurations. The tissue care devices can be expandable and/or contractible. The tissue devices can have an increasable and/or decreasable outer perimeter.

### CROSS-REFERENCE TO RELATED APPLICATION

This application is a continuation of International Patent Application No. PCT/US2019/048347 filed Aug. 27, 2019, which claims priority to U.S. Provisional Application No. 62/732,979 filed Sep. 18, 2018, each of which is herein incorporated by reference in its entirety for all purposes.

### BACKGROUND

### 1. Technical Field

Tissue care devices and methods of using the same are disclosed. More specifically, expandable and/or contractible tissue care devices and methods of using the same are disclosed.

### 2. Background of the Art

A need exists for improved tissue care devices that can accelerate healing, increase user comfort, reduce scarring and provide reliable tissue traction.

### BRIEF SUMMARY

This disclosure relates generally to tissue care devices.

More specifically, tissue closure devices and methods of using the same are disclosed. The devices can be expandable, contractible, or both.

Tissue care devices (e.g., wound care devices, pustule treatment devices) are disclosed. For example, a tissue care device is disclosed having a device longitudinal axis. The device can have tissue engagers. The device can have mounts coupled to the tissue engagers. The device can have a contractible band coupled to the mounts.

Tissue care devices (e.g., wound care devices, pustule treatment devices) are disclosed. For example, a tissue care device is disclosed having a device longitudinal axis. The device can have tissue engagers. The device can have a contractible band coupled to the tissue engagers.

Methods for deploying tissue care devices (e.g., wound care devices, pustule treatment devices) are disclosed. For example, a method is disclosed for deploying a tissue care device having a device longitudinal axis. The method can include expanding the device. The method can include attaching the device to tissue having a wound. The method can include contracting the device to partially or entirely close the wound.

### BRIEF DESCRIPTION OF THE DRAWINGS

The drawings shown and described are exemplary variations and non-limiting. Like reference numerals indicate identical or functionally equivalent features throughout.

FIGS. 1A and 1B illustrate a variation of a tissue care device and a method for expanding and contracting the device.

FIG. 1A illustrates the tissue care device in a device first configuration (e.g., a device contracted configuration).

FIG. 1B illustrates the tissue care device of FIG. 1A in a device second configuration (e.g., a device expanded configuration).

FIGS. 2A and 2B illustrate a variation of a tissue care device and a method for expanding and contracting the device.

FIG. 2A illustrates the tissue care device in a device first configuration (e.g., a device contracted configuration).

FIG. 2B illustrates the tissue care device of FIG. 2A in a device second configuration (e.g., a device expanded configuration).

FIGS. 3A and 3B illustrate a variation of a tissue care device and a method for expanding and contracting the device.

FIG. 3A illustrates the tissue care device in a device first configuration (e.g., a device contracted configuration).

FIG. 3B illustrates the tissue care device of FIG. 3A in a device second configuration (e.g., a device expanded configuration).

FIGS. 4A and 4B illustrate a variation of a tissue care device and a method for expanding and contracting the device.

FIG. 4A illustrates the tissue care device in a device first configuration (e.g., a device contracted configuration).

FIG. 4B illustrates the tissue care device of FIG. 4A in a device second configuration (e.g., a device expanded configuration).

FIGS. 5A and 5B illustrate a variation of a tissue care device and a method for expanding and contracting the device.

FIG. 5A illustrates the tissue care device in a device first configuration (e.g., a device contracted configuration).

FIG. 5B illustrates the tissue care device of FIG. 5A in a device second configuration (e.g., a device expanded configuration).

FIGS. 6A and 6B illustrate a variation of a tissue care device and a method for expanding and contracting the device.

FIG. 6A illustrates the tissue care device in a device first configuration (e.g., a device contracted configuration).

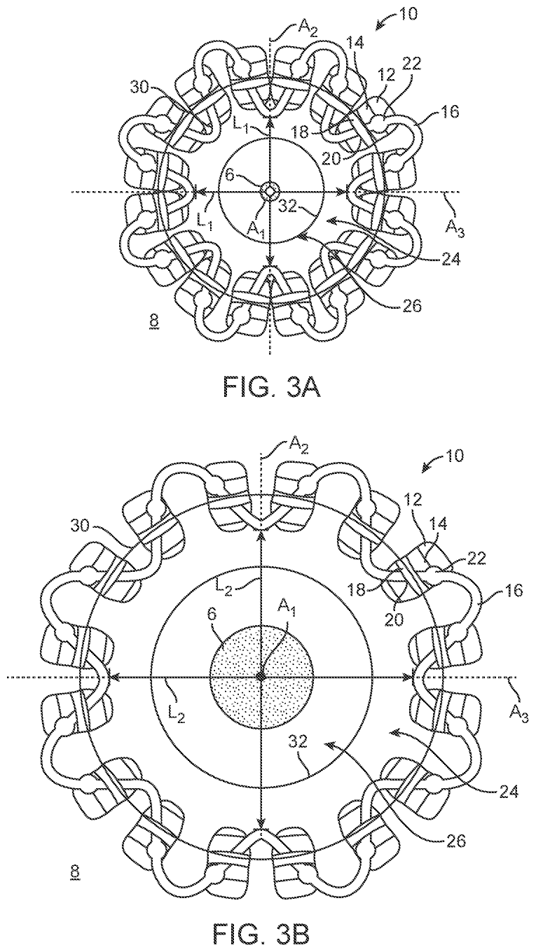

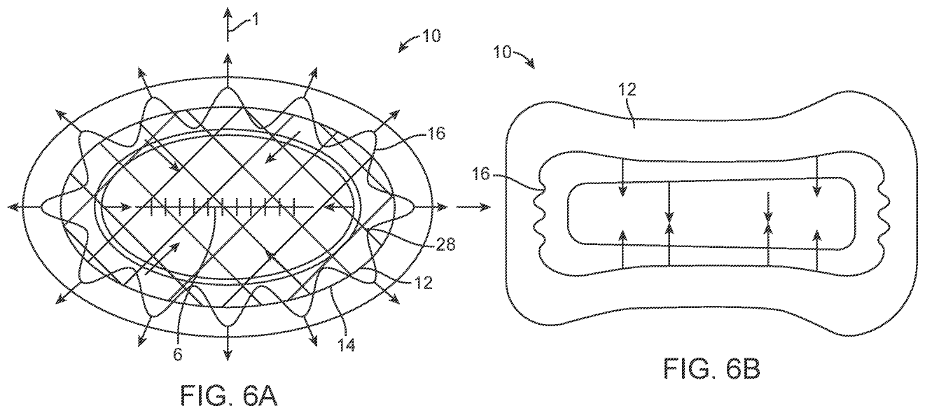

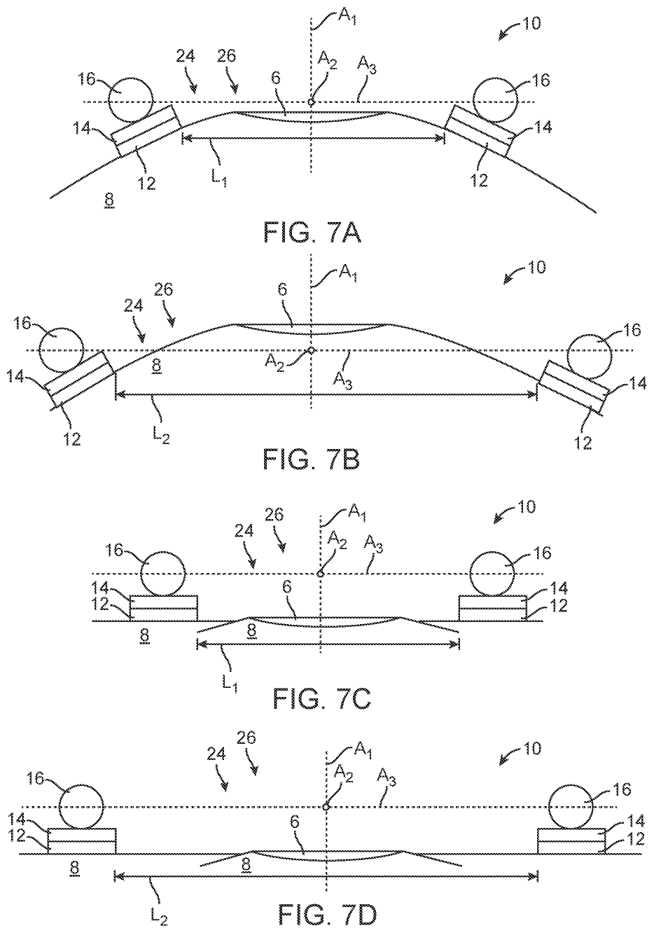

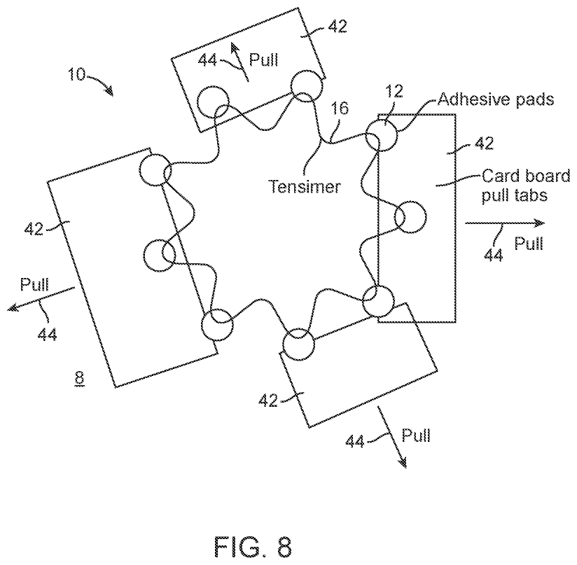

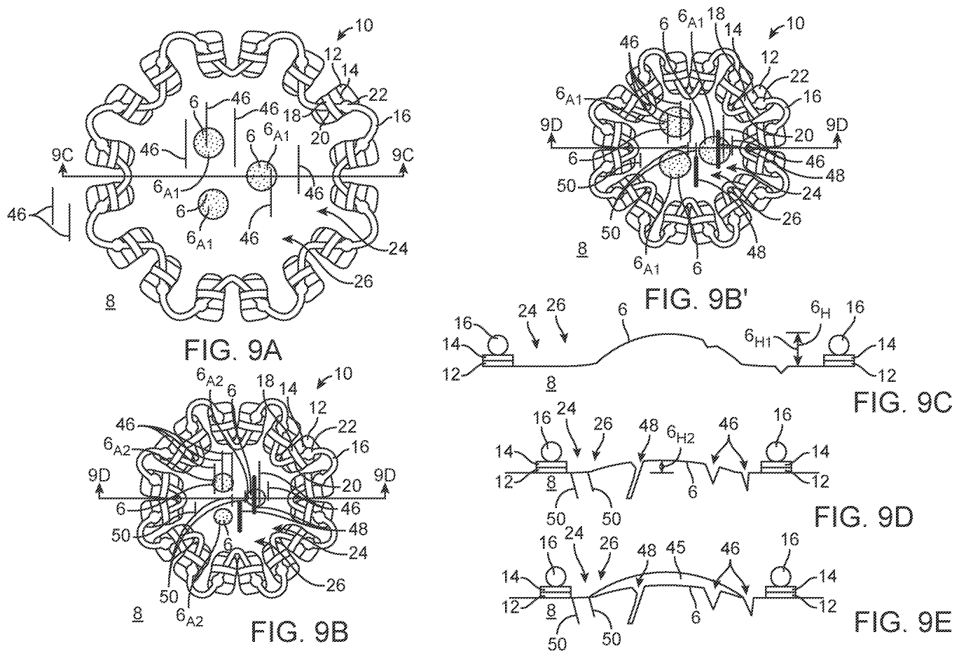

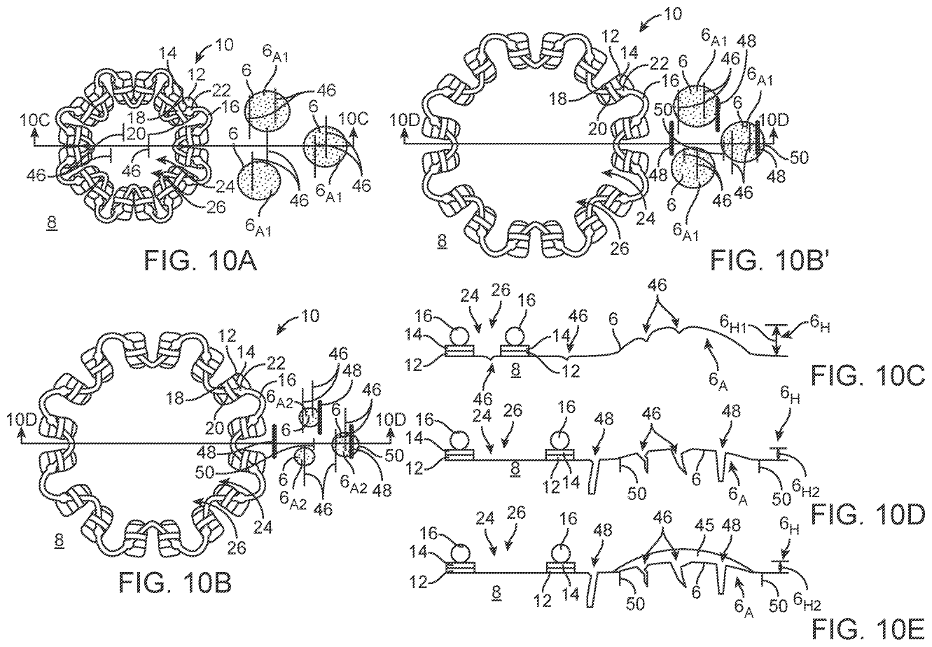

FIG. 3B illustrates the tissue care device of FIG. 6A in a device second configuration (e.g., a device expanded configuration).

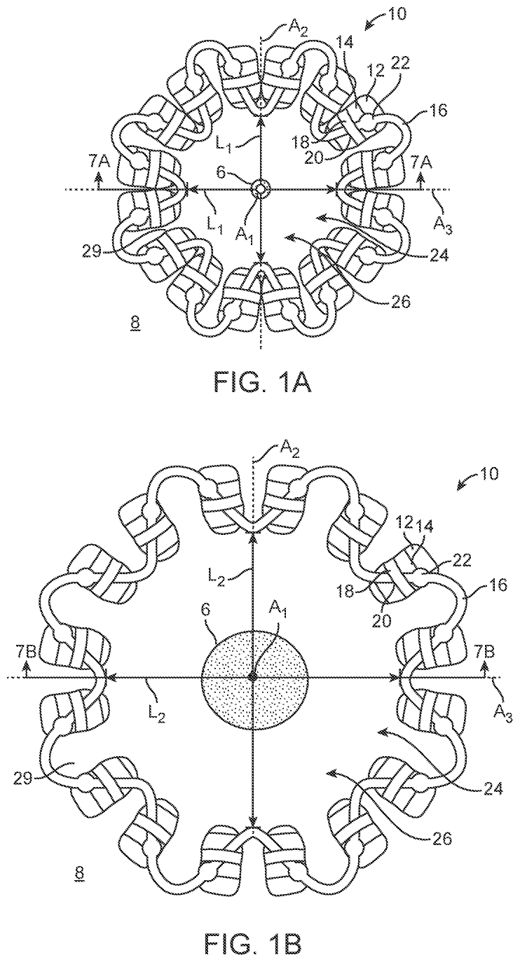

FIG. 7A illustrates the device of FIG. 1A taken along line 7A-7A with the device on a curved tissue surface.

FIG. 7B illustrates the device of FIG. 1B taken along line 7B-7B with the device on a curved tissue surface.

FIG. 7C illustrates the device of FIG. 1A taken along line 7A-7A with the device on a flat tissue surface.

FIG. 7D illustrates the device of FIG. 1B taken along line 7B-7B with the device on a flat tissue surface.

FIG. 8 illustrates a variation of a tissue care device and a method for expanding and contracting the device.

FIGS. 9A-9E illustrate a variation of a tissue care device and a method for treating tissue.

FIG. 9A illustrates the tissue care device in a device first configuration (e.g., a device expanded configuration).

FIG. 9B illustrates the tissue care device of FIG. 9A in a device second configuration (e.g., a device contracted configuration).

FIG. 9B′ illustrates the tissue care device of FIG. 9A in a device second configuration (e.g., a device contracted configuration).

FIG. 9C illustrates the device of FIG. 9A taken along line 9C-9C with the device on a flat and/or a curved surface.

FIG. 9D illustrates the device of FIG. 9B taken along line 9D-9D with the device on a flat and/or a curved surface.

FIG. 9E illustrates the device of FIG. 9D a solution applied to a surface of the tissue.

FIGS. 10A-10E illustrate a variation of a tissue care device and a method for treating tissue.

FIG. 10A illustrates the tissue care device in a device first configuration (e.g., a device contracted configuration).

FIG. 10B illustrates the tissue care device of FIG. 10A in a device second configuration (e.g., a device expanded configuration).

FIG. 10B′ illustrates the tissue care device of FIG. 10A in a device second configuration (e.g., a device expanded configuration).

FIG. 10C illustrates the device of FIG. 10A taken along line 10C-10C with the device on a flat and/or a curved surface.

FIG. 10D illustrates the device of FIG. 10B taken along line 10D-10D with the device on a flat and/or a curved surface.

FIG. 10E illustrates the device of FIG. 10D a solution applied to a surface of the tissue.

### DETAILED DESCRIPTION

Tissue care devices are disclosed. The tissue care devices can be and/or can function as a wound closer, a wound opener, a tissue closer, a tissue opener, a tissue tensioner, a tissue contractor, a tissue treater, or any combination thereof. For example, as a tissue treater, the devices can be and/or can function as a wound care device, as a pustule care device, as a lesion care device, or any combination thereof. The devices can treat tissue. The treated tissue can include wounds, pustules (e.g., acne), or both. For example, the tissue care devices can close wounds, open wounds, or both. The wounds can be open wounds, for example, cuts, incisions, lacerations, abrasions, avulsions, punctures, or any combination thereof. As another example, the tissue care devices can stretch tissue, compress tissue, or both. When the devices stretch or compress tissue, tension near tissue of one or multiple pustules (e.g., acne) can be reduced, eliminated, or both. The tissue care devices can be directly or indirectly anchored to tissue. The tissue care devices can partially or entirely close a lesion, a pustule, a wound, or any combination thereof. The tissue care devices can partially or entirely open a lesion, a pustule, a wound, or any combination thereof. The tissue care devices can partially or entirely relieve tissue pressure near one or multiple tissue pustules or lesions (e.g., acne). The tissue care devices can de-stretch or de-tension tissue over and/or near one or multiple tissue pustules or lesions (e.g., acne).

The tissue care devices can be expandable, contractible, or both. The tissue care devices can be expanded. The tissue care devices can be contracted. The tissue care devices can move skin and underlying tissue when expanded. The tissue care devices can move skin and underlying tissue when contracted. The tissue care devices can move skin and underlying tissue when expanded and contracted. For example, when the tissue care devices are attached to tissue, the tissue care devices can close wounds by contracting, open wounds by expanding, or both. As another example, when the tissue care devices are attached to tissue, the tissue care devices can relieve pressure on and/or near pustules by urging (e.g., pushing, pulling) more tissue toward the pustules. When the device is contracted or expanded, the tissue care devices can tension tissue, compress tissue, or both. The tissue care devices can maintain wounds in a closed or open state, for example, when the device is in a contracted or expanded configuration, respectively. The tissue care devices can maintain tissue near a pustule in a less tensioned condition so that the skin and tissue associated with and near the pustule is less stretched, which can advantageously reduce scarring caused by pustules (e.g., acne). The tissue care devices can maintain tissue in a state of traction. The traction can include tension and/or compression of tissue, of a wound, of a pustule, or any combination thereof. The tissue care devices can traction tissue to close wounds. This can allow the wound to heal. Maintaining the tissue in a desirable healing position can advantageously decrease the healing time and the amount of scar tissue that is formed.

### System and Apparatus

FIGS. 1A and 1B illustrate a variation of a tissue care device 10 that can be removably attached to a medium 8. The medium 8 can be organic (e.g., tissue), inorganic (e.g., fabric), or both. For example, the medium 8 can be skin, a tissue dressing, or both. The tissue dressing can be a wound dressing. The tissue dressing can have an absorbent portion, for example, an absorbent layer. The tissue dressing can have a drug-eluting portion, for example, a drug-eluting layer integrated with and/or separate from the absorbent layer. The tissue dressing can be a pustule dressing. The medium 8 can have target tissue 6. The target tissue 6 can be one or more wounds, lesions, pustules (e.g., acne), or any combination thereof. The device 10 can be positioned over the target tissue 6. The device 10 can be positioned partially or entirely around the target tissue 6. The device 10 can extend partially or entirely around the target tissue 6.

The device 10 can have one or more engagers 12, one or more mounts 14 and one or more bands 16. The engagers 12 can attach the device 10 to the medium 8. The mounts 14 can couple the bands 16 to the engagers 12. The bands 16 can expand. The bands 16 can contract. The bands 16 can expand and contract. The device 10 can be expandable and contractible via the bands 16. For example, the bands 16 can be expanded and contracted to expand and contract the device 10, respectively.

The engagers 12 can anchor the device 10 to the medium 8 with or without piercing the medium 8. The engagers 12 can have one or more mechanical fasteners, chemical fasteners, or both. For example, the engagers 12 can have an adhesive. The device 10 can be attached to the medium 8 with the adhesive. The engagers 12 can be adhesive patches. Additionally or alternatively, the engagers 12 can include one or more piercers (e.g., staples), magnets, hooks, snaps, male connectors, female connectors, or any combination thereof. For example, the engagers 12 can have a first fastener (e.g., adhesive) and a second fastener (e.g., adhesive, hook), where the first fastener can attach the engager 12 to tissue and the second fastener can attach the engager 12 to a tissue dressing (e.g., a wound dressing). The engagers 12 can be separate from a tissue dressing or can be attached to or integrated with a tissue dressing. The engagers 12 can be flexible, rigid, or both. The engagers 12 can be deformable.

The engagers 12 can have an engager first portion and an engager second portion. The engager first portion can be removably attached to the medium 8. When attached to the medium 8, the engager first portion can be in direct or indirect contact with the medium 8, for example, via an adhesive or a mechanical fastener. The engager second portion can be attached to or integrated with one or more of the mounts 14, for example, via a chemical or mechanical fastener. The engager first and second portions can be flexible, rigid, or both. For example, the engager first portion can be flexible to allow the device 10 to conform to the surface of the medium 8 and the engager second portion can be rigid to allow for the band 16 to be removably attached to the mounts 14.

The engager first and second portions can be opposite one another (e.g., diametrically opposite one another). For example, the engager first portion can be on an engager first side (e.g., an engager bottom) and the engager second portion can be on an engager second side (e.g., an engager top) opposite the engager first side. The engager first portion can contact tissue and the engager second portion can have a mount 14.

The engager first and second portions can be adjacent to one another. For example, the engager first portion can be on an engager first side (e.g., an engager bottom) and the engager second portion can be on an engager second side next to the engager first side. The engager second side can be a side of the engager between the top and bottom sides of the engager, for example, the side closer to a device center, the size farther from the device center, or the sides extending therebetween.

The mounts 14 can couple the bands 16 to the engagers 12, for example, with or without one or more couplers 18. The couplers 18 can contact the bands 16. For example, the couplers 18 can wrap around a portion of the bands 16. The couplers 18 can be attached to or integrated with the mounts 14. The mounts and/and couplers 14, 18 can define one or more channels 20. The bands 16 can be fixed in the channels 20, can be movable in the channels 20, or both. For example, the bands 16 can move in the channels 20 when the device 10 gets smaller (e.g., when the device 10 changes from a device expanded configuration to a device contracted configuration), when the device 10 gets larger (e.g., when the device 10 changes from a device contracted configuration to a device expanded configuration), or both. For example, the bands 16 can be translatable (e.g., slideable) in the channels 20, rotatable in in the channels 20, or both. The bands 16 can be removably or irremovably attached to channels 20.

The mounts and/or couplers 14, 18 can define one or more grooves for the bands 16 to sit in (e.g., on the surface of the mounts 14, on the surface of the couplers 18, or both). The mounts and/or couplers 14, 18 can define one or more grooves for the bands 16 to move in (e.g., on the surface of the mounts 14, on the surface of the couplers 18, or both). For example, the bands 16 can be translatable (e.g., slideable) in the grooves, rotatable in the grooves, or both. The couplers 18 can be straps, hooks, magnets, or any combination thereof. As another example, the bands 16 can be attached to or integrated with the engagers 12 without the mounts 14. The engagers 12 can define the channels 20 and grooves.

The mounts 14 can allow, inhibit, or prevent the bands 16 from moving relative to the mounts 14. For example, the bands 16 can be attached to the mounts 14 with a loose fit, restrictive fit, friction fit, snap fit, magnetic fit, glue fit, or any combination thereof. The bands 16 can be slideable in the mounts 14, for example, in the channels 20 or in the grooves. The mounts 14 can be flexible, rigid, or both. The mounts 14 can be deformable. For example, the engagers 12 can be flexible and the mounts 14 can be rigid.

The engagers and mounts 12, 14 can be modular such that the engagers and mounts 12, 14 can be removably attached to one another. For example, the engagers 12 can be removably attached to the mounts 14, or vice versa. The engagers and mounts 12, 14 can be modular such that the engagers and mounts 12, 14 can be removably attached to the bands 16. For example, the engagers 12 can be removably attached to the bands 16. As another example, the mounts 14 can be removably attached to the bands 16. As yet another example, the engagers and mounts 12, 14 can be removably attached to the bands 16. This can desirably allow different numbers of engagers and mounts 12, 14 to be used with the same band 16, for example, to accommodate target tissues 6 having different shapes and sizes. As another example, bands 16 having different sizes (e.g., small, medium, large bands) can be used with the device 10 to accommodate target tissues 6 having different shapes and sizes. This can desirably allow the device 10 to be used with target tissues 6 having different sizes and shapes, and can desirably allow the device 10 to treat the same target tissue 6 as it heals and becomes smaller. The bands 16 can have different shapes to accommodate different wound shapes. The band shape can be shaped to follow the contour of the target tissue 6. The band shape can mirror the shape of the target tissue 6, for example, round bands 16 for round target tissues 6, square bands 16 for square target tissues 6, irregular shaped bands 16 for irregular shaped target tissues.

The bands 16 can be attached to the engagers and/or mounts 12, 14 on the side opposite the medium 8, for example, on a top surface of the engagers and/or mounts 12, 14. As another example, the bands 16 can be attached to the engagers and/or mounts 12, 14 on one or more side surfaces of the engagers and/or mounts 12, 14, where the side surfaces can be between the top and bottom surfaces of the engagers and/or mounts 12, 14. The engagers and/or mounts 12, 14 can have an outer side surface (e.g., the surface farthest from the device center), an inner side surface (e.g., the surface closest to the device center) and intermediate side surfaces extending therebetween. The bands 16 can be attached to or integrated with any of these surfaces.

The bands 16 can be expandable, stretchable, contractible, compressible, foldable, unfoldable, rotatable, translatable, telescopable, or any combination thereof. For example, the bands 16 can be non-stretchable but can fold and unfold when the bands 16 decrease and increase in size (e.g., diameter, maximum length across the device in any device configuration), respectively. As another example, the bands 16 can be stretchable and can fold and unfold when the bands 16 decrease and increase in size, respectively. The bands 16 can have a fixed length, a changeable length, or both. The bands 16 can expand, stretch, contract, compress, fold, unfold, rotate, translate, telescope, or any combination thereof, for example, in response to an internal force, an external force (e.g., a force applied to the device 10), or both. The internal force can be an internal spring force of the bands 16. The internal force can be internal to the device 10 but external to the bands 16. For example, the device 10 can have a spring which imparts a force to the bands 16. The external force can be applied directly or indirectly via a person's hands. The external force can be applied via a tool. The tool can be removably engageable with the device 10. The bands 16 can expand, stretch, contract, compress, fold, unfold, rotate, translate, telescope, or any combination thereof, for example, relative to any portion of the device 10, including for example, relative to a center of the device 10, relative to any one of the engagers 12 or mounts 14, relative to any group of engagers 12 or mounts 14, or any combination thereof.

The bands 16 can be made of plastic, metal, composite material, or any combination thereof. For example, the bands 16 can be made of a shape memory alloy (e.g., nitinol). As another example, the bands 16 can be made of a non-shape memory material (e.g., a non-shape memory alloy). The bands 16 can be elastic, inelastic, or both. The bands 16 can be springs. The bands 16 can be made of one or multiple filaments. The filaments can be made from one or multiple materials. For bands 16 with multiple filaments, the bands 16 can be braided or non-braided. Different filaments can be made of the same or different material as another filament. For example, the bands 16 can have a first filament and a second filament. The first filament can be made of a first material and the second filament can be made of a second material the same as or different from the first filament. Where the first and second materials are different from one another, the first material can be elastic and the second material can be inelastic, the first material can be more elastic than the second material, the first material can be less elastic than the first material, or any combination thereof. The first and second filaments can be braided together. A filament can have multiple longitudinal segments. The segments can be removably or permanently attached together. Different segments can be made of the same or different material as another filament. For example, the bands can have multiple first segments and multiple second segments. The first segments and second segments can alternate with on another such that a first segment (e.g., each first segment) is between two second segments. The multiple first segments can be made of a first material and the multiple second segments can be made of a second material the same as or different from the multiple first segments. Where the first and second materials are different from one another, the first material can be elastic and the second material can be inelastic, the first material can be more elastic than the second material, the first material can be less elastic than the first material, or any combination thereof. The bands 16 can be an expandable column, for example, made of struts and open cells. For example, the struts can define an expandable and contractible toroid. The toroid can be closed (e.g., forming a ring) or open (e.g., forming an arc or polyline shape). As another example, the bands 16 can form a helix. The helix can form a closed or open toroid. The bands 16 can be an expandable cage. As another example, the bands 16 can be made of multiple links. The links can be connected to each other via flexible or rigid joints. As another example, each link can be connected to one or multiple engagers 12 and/or to one or multiple mounts 14 with or without being connected to another link. The links can have a uniform size. There can be links of different sizes. Some or all of the links can be flexible. Some or all of the links can be rigid. Some links can be more flexible than other links. For example, a first link can be more flexible than a second link. Some links can be more rigid than other links. For example, a first link can be more rigid than a second link. The links can be connected together to form a band 16, for example, a circular or polygonal band 16. The links can be straight, curved, bent, or any combination thereof. The links can be connected to form a polyline band 16. The links can be connected to form an expandable cage. The links can be struts of an expandable cage.

The bands 16 can be shape set in a closed configuration (also referred to as a contracted configuration). The bands 16 can be shape set in an open configuration (also referred to as an expanded configuration). The shape set configuration of the bands 16 can be the neutral configuration of the bands 16. The bands 16 can be in a strained configuration when not in their neutral or shape set configuration. The bands 16 can be biased to automatically return to or return toward their neutral configuration when expanded and/or contracted away from the neutral configuration. For example, the bands 16 can be biased to automatically return to or return toward their neutral configuration after being expanded and/or contracted away from the neutral configuration when the expansion and/or contraction force, respectively, is released from the device 10 (e.g., released from the band 16). As another example, the bands 16 can automatically return to or return toward their neutral configuration after being expanded and/or contracted away from the neutral configuration when the expansion and/or contraction force, respectively, is released from the device 10 (e.g., released from the band 16).

All or a portion of the bands 16 can be shape set. The bands 16 need not be shape set. All or a portion of the bands 16 can be not shape set. One or more portions of the bands 16 can be shape set. One or more portions of the bands 16 need not be shape set. The bands 16 can have one or more portions that are shape set and one or more portions that are not shape set. For example, a band first portion can be shape set and a band second portion can be not shape set.

The bands 16 can be expanded and/or contracted with or without the use of a tool. The device 10 can be expanded and/or contracted with one hand, for example, via the bands 16. The device 10 can be expanded and/or contracted with two hands. The device 10 can be expanded and/or contracted with one and/or or two hands.

When the bands 16 expand or contract, the bands 16 can change from a band first configuration to a band second configuration different from the band first configuration. The band second configuration can be more open than the band first configuration. The band second configuration can be more closed than the band first configuration. The band first configuration can be a fully contracted configuration, a partially contracted configuration, a partially expanded configuration, or a fully expanded configuration. The band second configuration can be a fully contracted configuration, a partially contracted configuration, a partially expanded configuration, or a fully expanded configuration. The bands 16 can change configurations (e.g., from the band first configuration to the band second configuration, or vice versa) by folding, unfolding, stretching, compressing, or any combination thereof. For example, the bands 16 can unfold and/or stretch when the bands 16 change from a first configuration to a second configuration more open than the first configuration. As another example, the bands 16 can fold and/or compress when the bands 16 change from a first configuration to a second configuration less open than the first configuration. For example, FIGS. 1A and 1B illustrate that the band 16 can have a band first configuration (FIG. 1A) and a band second configuration (FIG. 1B), where the band second configuration is more open than the band first configuration.

The device 10 can have a device first configuration (e.g., FIG. 1A) and a device second configuration (e.g., FIG. 1B), for example, when the band 16 is in a band first configuration (e.g., FIG. 1A) and when the band 16 a band second configuration (e.g., FIG. 1B), respectively. The bands 16 can control the size of the device 10. The bands 16 can change the size of the device 10. The bands 16 can be manipulated to control the size of the device 10. The bands 16 can be manipulated to increase the size of the device, decrease the size of the device, or both. As another example, one or more of the engagers 12 and/or mounts 14 can be manipulated to increase or decrease the size of the device such that the bands 16 can expand and/or contract when the one or more engagers 12 and/or mounts 14 are manipulated.

The device first configuration can be a device neutral configuration, a device open configuration, a device expanded configuration, a device closed configuration, a device contracted configuration, or any combination thereof. The device neutral configuration can be a device non-expanded configuration, a device expanded configuration, a device non-contracted configuration, a device contracted configuration, or any combination thereof. The device second configuration can be a device neutral configuration, a device expanded configuration, a device contracted configuration, or any combination thereof. The device 10 can be changed from the device first configuration to the device second configuration. When the device 10 is changed from the device first configuration to the device second configuration, the device 10 can increase in size or decrease in size. The device 10 can be changed from the device second configuration to the device first configuration. When the device 10 is changed from the device second configuration to the device first configuration, the device 10 can increase in size or decrease in size. For example, the size of the device 10 can be increased (e.g., the device 10 can be expanded) from a device neutral configuration, a device expanded configuration, a device contracted configuration, or any combination thereof. As another example, the size of the device 10 can be decreased (e.g., the device 10 can be contracted) from a device neutral configuration, a device expanded configuration, a device contracted configuration, or any combination thereof.

The device can have one or multiple neutral configurations. For example, the device neutral configuration can correspond to when the bands 16 are in their shape set shape. As another example, the device neutral configuration can correspond to when the bands 16 are in a relaxed state or in a relaxed state most permitted by the device 10. The relaxed state can correspond to a non-stretched state or a partially non-relaxed state (e.g., a partially stretched state). The device 10 can changeable from the device first configuration to the device second, from the device second configuration to the device first configuration, or both. For example, FIG. 1A illustrates the device 10 in a device neutral configuration and FIG. 1B illustrates the device 10 in a device non-neutral configuration. As another example, FIG. 1A illustrates the device 10 in a device non-neutral configuration and FIG. 1B illustrates the device 10 in a device neutral configuration. As yet another example, FIG. 1A illustrates the device 10 in a device non-neutral configuration and FIG. 1B illustrates the device 10 in a device non-neutral configuration.

The device can have a device center axis A₁. The device 10 can expand symmetrically or asymmetrically away from the device center axis A₁. For example, FIGS. 1A and 1B illustrate that the device 10 can expand symmetrically away from the device center axis A₁. When the device 10 is in the device first configuration (e.g., FIG. 1A), the engagers 12, the mounts 14 and/or the bands 16 can be a first distance away from the device center axis A₁. When the device 10 is in the device second configuration (e.g., FIG. 1B), the engagers 12, the mounts 14 and/or the bands 16 can be a second distance away from the device center axis A₁, where the second distance can be greater than the first distance. The target tissue 6 can be centered or offset relative to the device center axis A₁ when the device is in the device first configuration. The target tissue 6 can be centered or offset relative to the device center axis A₁ when the device is in the device second configuration. For example, FIGS. 1A and 1B illustrate that the device center axis A₁ can coincide with a central area of the target tissue 6 (e.g., with a center axis of the target tissue 6).

The device 10 can have a device first maximum dimension Li when the device 10 is in the device first configuration (e.g., FIG. 1A). The dimension L₁ can be a length across the device 10, for example, as measured between two points on an outer perimeter of the device 10, as measured between two points on an inner perimeter of the device 10, or as measured between any two points on opposite sides of the device 10 (e.g., between a point on a first engager 12, on a first mount 14, or on first portion of the bands 16 and a point on a second engager 12, on a second mount 14, or on a second portion of the bands 16, where the points are on opposite sides of the device 10). The dimension L₁ can be the maximum length across the device when the device 10 is in the device first configuration. The dimension L₁ can be, for example, about 1 cm to about 50 cm, including every 1 cm increment within this range (e.g., 2 cm, 4 cm). The dimension L₁ can be a diameter of the device 10. The dimension L₁ can be, for example, about 1 cm to about 50 cm, including every 1 cm increment within this range (e.g., 1 cm, 2 cm, 10 cm). FIG. 1A illustrates, for example, that the device 10 can have the dimension L₁ along a device first transverse axis A₂ and along a device second transverse axis A₃.

The device 10 can have a device second maximum dimension L₂ when the device 10 is in the device second configuration (e.g., FIG. 1B). The dimension L₂ can be a length across the device 10, for example, as measured between two points on an outer perimeter of the device 10, as measured between two points on an inner perimeter of the device 10, or as measured between any two points on opposite sides of the device 10 (e.g., between a point on a first engager 12, on a first mount 14, or on first portion of the bands 16 and a point on a second engager 12, on a second mount 14, or on a second portion of the bands 16, where the points are on opposite sides of the device 10). The dimension L₂ can be the maximum length across the device when the device 10 is in the device second configuration. The dimension L₂ can be, for example, about 1 cm to about 50 cm, including every 1 cm increment within this range (e.g., 5 cm, 10 cm, 15 cm). The dimension L₂ can be a diameter of the device 10. The dimension L₂ can be, for example, about 1 cm to about 50 cm, including every 1 cm increment within this range (e.g., 1 cm, 2 cm, 10 cm). FIG. 1B illustrates, for example, that the device 10 can have the dimension L₂ along the device first transverse axis A₂ and along the device second transverse axis A₃.

The length of the bands 16 can be the same or different in the contracted and expanded configurations. For example, the bands 16 can have the same length in the expanded configuration than in the contracted configuration (e.g., where the bands 16 can fold and unfold). As another example, the bands 16 can have a greater length in the expanded configuration than in the contracted configuration (e.g., where the bands 16 can stretch and de-stretch).

The diameter of the bands 16 can be the same or different in the contracted and expanded configurations. For example, the bands 16 can have the same diameter in the expanded configuration as in the contracted configuration (e.g., where the bands 16 fold and unfold). As another example, the bands 16 can have a greater diameter in the expanded configuration than in the contracted configuration (e.g., where the bands 16 can stretch and de-stretch).

The bands 16 can have a band longitudinal axis that follows the shape of the bands 16. The band longitudinal axes can be a center longitudinal axis of the bands 16. The bands 16 can be straight and/or curved. The bands 16 can have a zig-zag pattern (also referred to as an undulating pattern). The corners of the zig zag pattern can be sharp (e.g., pointed) or rounded. The zig zag pattern can have a sinusoidal shape (e.g., curved, square) having branches and corners. The branches can be rigid and the corners can be flexible. As another example, the corners can be more flexible than the branches. The bands 16 can undulate in one or multiple planes. FIGS. 1A and 1B illustrate that the bands 16 can have an undulating shape (e.g., a sinusoidal shape) having branches and corners, where each branch between adjacent corners of the band 16 can be attached to a mount 14.

FIG. 1A further illustrates that the band longitudinal axis along the branches can be at an alignment angle of about 0 degrees to about 45 degrees with a longitudinal axis of the engager 12, the mount 14, or both, including every 1 degree increment within this range (e.g., 5 degrees, 10 degrees, 15 degrees, 20 degrees) when the device 10 is in a closed state (also referred to as a contracted configuration). Such alignment angles can desirably improve the force transfer between the device 10 and the medium 8. FIG. 1B further illustrates that the band longitudinal axis along the branches can be at an alignment angle of about 0 degrees to about 45 degrees with a longitudinal axis of the engager 12, the mount 14, or both, including every 1 degree increment within this range (e.g., 5 degrees, 10 degrees, 15 degrees, 20 degrees) when the device 10 is in an open state (also referred to as an expanded configuration). Such alignment angles can desirably improve the force transfer between the device 10 and the medium 8. The alignment angle can be the same or different when the device 10 is in the contracted and expanded configurations. For example, the band longitudinal axis can be more closely aligned (e.g., by about 5 degrees, 10 degrees, 15 degrees) with the longitudinal axis of the engager 12, the mount 14, or both when the device 10 is in a contracted configuration (e.g., fully contracted configuration) than when the device 10 is in an expanded configuration (e.g., fully expanded configuration).

The bands 16 can have bulges 22 configured to engage with the engagers 12, the mounts 14, or both. The bulges 22 can be attached to or integrated with the bands 16. The bulges 22 can extend away from the band longitudinal axis. The bulges 22 can have spherical shape, a pearl shape, a bead shape, or any combination thereof. The bulges 22 can be a ramp. The bulges 22 can be a hook. The bulges 22 can be one or more extensions away from the bands 16. The bulges 22 can allow for the controllable opening and/or closing of the device 10, for example, by inhibiting or preventing the bands 16 from slipping through the engagers and/or mounts 12, 14 when the device 10 changes from an expanded configuration to a contracted configuration, or vice versa. For example, the bulge 22 can be sized to not pass through the channel 20 such that the bulge 22 can push against the mount 14 when the device 10 closes or opens. When the bulges 22 are pushed against the mounts 14, the bulges 22 can become attached to the mounts 14 (e.g., in the channel 20) with a friction fit, snap fit, magnetic fit, or any combination thereof.

The bulges 22 can be arranged uniformly on the band 16 around the device 10, for example, for uniformly shaped target tissues 6 (e.g., target tissues having symmetry across two perpendicular axes). The bulges 22 can be arranged non-uniformly (e.g., in a pattern or irregularly) on the band 16 around the device 10, for example, for irregular shaped target tissues 6 (e.g., target tissues having no symmetry or symmetry across one axis).

The bands 16 can be closed (e.g., forming a loop) or open (e.g., forming an arc or polyline shape). For example, the bands 16 can be rings (e.g., nitinol rings). For closed devices 10 where the bands 16 form a loop, the one or more bands 16 forming the loop can define an opening 24 of the device 10. The opening 24 can be a central opening of the device 10. The opening 24 can have a circular, elliptical, polygonal (e.g., diamond), and/or irregular shape. For open devices 10 where the one or more bands 16 do not form a loop, the opening 24 can be a cavity. The cavity can have a shape that is a partial circle, ellipse, polygon (e.g., diamond), irregular shape, or any combination thereof. The opening 24 can desirably allow the target tissue 6 to breath during the healing process. The bands 16 can have a regular or irregular cross-sectional shape, for example, circular, elliptical, stadium, polygonal (e.g., diamond-shaped, star-shaped), or any combination thereof.

The device 10 can have an opening 26. The opening 26 can be the same as or different from the opening 24. The opening 26 can be a center opening of the device 10. The opening 26 can expose the target tissue 6 when the bands 16 are in the expanded and/or contracted configurations. The opening 26 can desirably allow the target tissue 6 to breath during the healing process.

The openings 24 and 26 can increase and decrease in size with the device 10 is expanded and contracted, respectively. The openings 24 and/or 26 can desirably enable the target tissue 6 to be accessed when the device 10 is attached to the medium 8. The openings 24 and 26 are also referred to as tissue access ports. The tissue access port (e.g., opening 24, opening 26) can have a port area of about 1 cm² to about 1000 cm² or more, including every 1 cm² increment within this range. For example, the tissue access port can have a port area of about 30 cm² when the device 10 is in an expanded configuration and can have a port area of about 10 cm² when the device 10 is in a contracted configuration.

The bands 16 can be deformable, for example, resiliently deformable. The bands 16 can be deformed into one or multiple configurations. The bands 16 can deform to close the target tissue 6, open the target tissue 6, or both. For example, the bands 16 can expand and contract when the device 10 is attached to the medium 8.

FIGS. 1A and 1B further illustrate that there can be gaps 29 or channels 29 between adjacent engagers and/or mounts 12, 14. These can advantageously allow the target tissue 6 or a wound dressing to more breathe. This can be especially helpful where a cover is placed over the target tissue access ports 24, 26. FIG. 1A illustrates that some of the gaps 29 can be closed when the device 10 is in a closed configuration. As another example, all of the gaps 29 can be open when the device 10 is in a closed configuration. FIG. 1B illustrates that all of the gaps 29 can be open when the device is in an open configuration.

FIGS. 1A and 1B further illustrate that the gaps or channels between adjacent engagers and/or mounts 12, 14 can be larger in a device first configuration (e.g., FIG. 1B) than in a device second configuration (e.g., FIG. 1A) that is less expanded or more contracted than the device first configuration. For example, a dimension (e.g., length) between adjacent engagers and/or mounts 12, 15 can range from about 2 mm to about 20 mm in the device first configuration and can range from about 0 mm to about 18 mm in the device second configuration.

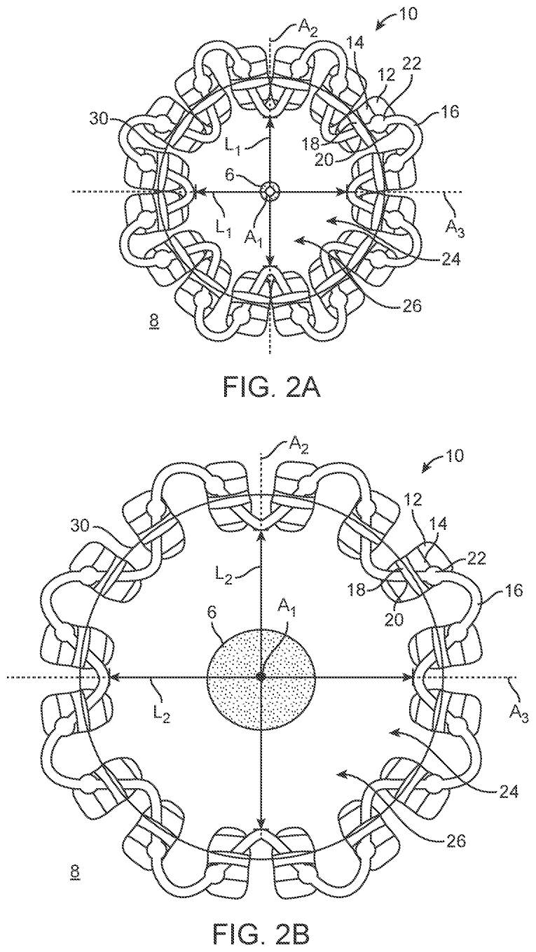

FIGS. 2A and 2B illustrate that the device 10 can have a cover 30 (also referred to as a tissue cover). The cover 30 can be removably attached to the engagers, mounts and/or bands 12, 14, 16. For example, FIGS. 2A and 2B illustrate that the cover 30 can be removably attached to the mounts 14. The cover 30 can partially or entirely cover the openings 24 and/or 26. The cover 30 can advantageously protect the target tissue 6 from becoming contaminated. The cover 30 can have a flap 32 which allows access to the target tissue 6. As another example, an edge of the cover 30 can be removably attachable to allow access to the target tissue 6. The cover 30 can be a filter. For example, the cover 30 can be an air filter, a wound exudate filter, or both. A side of the cover 30 (e.g., the side configured to face toward tissue) can be a wound dressing and/or can have a wound dressing for the target tissue 6. The wound dressing can be configured to contact the target tissue 6. When the device is in the device first configuration (e.g., FIG. 2A), the cover 30 and/or the wound dressing can contact the target tissue 6. When the device is in the device second configuration (e.g., FIG. 2B), the cover 30 and/or the wound dressing can contact the wound. The cover 30 and/or the wound dressing can be replaced or interchanged with a second cover and/or wound dressing, for example, after a pre-determined treatment period, after the cover and/or wound dressing becomes saturated with wound exudate, or after the cover and/or wound dressing become unsanitary. The cover 30 can be translucent. The cover 30 can be transparent. The cover 30 can be opaque.

FIGS. 3A and 3B illustrate that the cover 30 can have a flap 32. A side of the cover 30 (e.g., the side configured to face toward tissue) can be a wound dressing and/or can have a wound dressing for the target tissue 6. A side of the flap 32 (e.g., the side configured to face toward tissue) can be a wound dressing and/or can have a wound dressing for the target tissue 6. The wound dressing can be configured to contact the target tissue 6. When the device is in the device first configuration (e.g., FIG. 3A), the cover 30, the flap 32 and/or the wound dressing can contact the target tissue 6. When the device is in the device second configuration (e.g., FIG. 3B), the cover 30, the flap 32 and/or the wound dressing can contact the wound. The cover 30, the flap 32 and/or the wound dressing can be replaced or interchanged with a second cover, a second flap, and/or a second wound dressing, for example, after a pre-determined treatment period, after the cover, the flap and/or wound dressing becomes saturated with wound exudate, or after the cover, the flap and/or wound dressing become unsanitary. The flap 32 can be translucent. The flap 32 can be transparent. The flap 32 can be opaque.

The cover 30 can be expandable, non-expandable or both. The flap 32 can be expandable, non-expandable or both. The cover 30 and/or the flap 32 can be made of an elastic material such that they can conform to the size of the device 10 when the device 10 is in the device first configuration and when the device 10 is in the device second configuration.

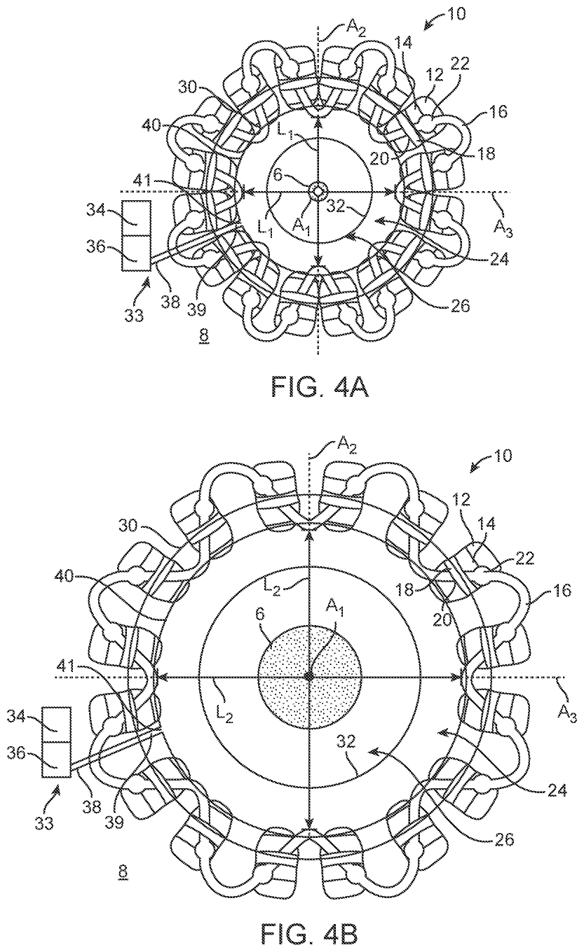

FIGS. 4A and 4B illustrate that the device 10 can have a negative pressure wound therapy system 33. The negative pressure wound therapy system 33 can include a pump 34 and a wound exudate reservoir 36. The pump 34 can be battery powered. One or more tubes 38 can connect the target tissue 6 to the pump and the canister 34, 36. When negative pressure is applied to the target tissue 6 via the pump 34, wound exudate can be pumped from the target tissue 6 to the reservoir 36. The reservoir 36 can be removably attached to the device 10, for example, via the tubing 38. The reservoir 36 can be removably attached to the device 10, for example, to one or more of the engagers 12, to one or more of the mounts 14, to the bands 16, or any combination thereof. The tubing 38 can have a one-way flow valve 39 to permit flow out of the wound but not into the wound. As another example, the tubing 38 does not have a one-way flow valve 39. As yet another example, the device 10 does not have a one-way flow valve 39.

FIGS. 4A and 4B further illustrate that the device 10 can have one or multiple skirts 40 configured to close off the wound from the outside environment in conjunction with the cover 30. The device 10 can have a skirt 40 between every two adjacent engagers and mounts 12, 14. As another example, the device 10 can have a single skirt 40 that runs along an inner perimeter of the device 10, for example, as shown in FIGS. 4A and 4B. The skirt 40 can have a port 41 for the tubing 38. The device 10 can have one or multiple skirts 40 with or without the negative pressure wound therapy system 33. The skirt 40 can be translucent. The skirt 40 can be transparent. The skirt 40 can be opaque. The skirt 40 can be expandable, non-expandable or both. The skirt 40 can be made of an elastic material so that it can conform to the size of the device 10 when the device 10 is in the device first configuration and when the device 10 is in the device second configuration.

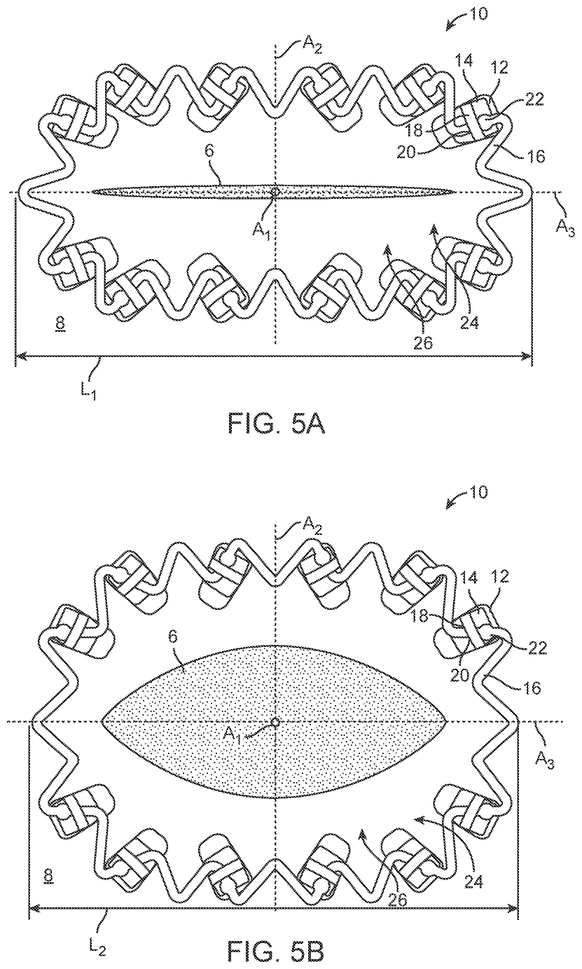

FIGS. 5A and 5B illustrate that the bands 16 can have an undulating shape (e.g., sinusoidal shape) where some of the branches between adjacent corners of the band 16 can be attached to a mount 14 and where some of the branches between adjacent corners of the band 16 need not be attached to a mount 14. FIGS. 5A and 5B further illustrate that the device 10 can have a non-circular shape, for example, an oblong or stadium shape. This can be helpful, for example, where the target tissue 6 has a greater length than width or vice versa. The length or the width can be the longer dimension. For example, FIGS. 5A and 5B illustrate that the length can be greater than the width. The length of the target tissue 6 can be about a 1 cm to about 25 cm or more, including every 1 cm increment within this range (e.g., 4 cm). FIGS. 5A and 5B further illustrate that the longitudinal axes of the band branches can remain aligned with the longitudinal axes of the engagers and/or mounts 12, 14 when the device is in a contracted configuration, an expanded configuration, and therebetween.

FIG. 6A illustrates that the engager 12 can have an annular shape. The engager 12 can be the rim of a tissue dressing (e.g., of a wound dressing).

FIG. 6A further illustrates that the mount 14 can have an annular shape. For example, the mount 14 can be a wire loop.

FIG. 6A further illustrates that the device 10 can have tensioners 28 across the device 10. The tensioners 28 can be self-ratcheting cables. The tensioners 28 can maintain the device 10 in an open or closed state. The tensioners 28 can be attached to or integrated with the engagers and/or mounts 12, 14. The tensioners 28 can be removed from the device 10 after the device 10 is contracted the desired amount. The tensioners 28 can be removed, for example, after the device 10 is locked into a contracted configuration using one or more locking mechanisms. The device 10 can remain in the contracted state after the tensioners 28 are removed. After the tensioners 28 are removed, they can be reattached to the device 10 to further contract or re-expand the device 10, after which the tensioners 28 can again be removed. The tensioners 28 can be integrated with or attached to the cover 30, the flap 32, the wound dressing, or any combination thereof.

FIG. 6B illustrates that the bands 16 can be less undulated in the expanded configuration than in the contracted configuration, or vice versa.

The device 10 can have ratchet components to keep the device 10 in an expanded or contracted state.

The device 10 can have one or multiple stops configured to inhibit or prevent the device 10 from over-contracting or over-expanding. The stops are also referred to as contraction limiters and expansion limiters. For example, the engagers and/or mounts 12, 14 can be configured to contact one another to prevent over-contraction of the device 10 when the device 10 is in a maximally contracted state. A first engager and/or mount 12, 14 can, for example, contact a second and/or a third engager and/or mount 12, 14 when the device 10 reaches a maximally contracted state. In this way, the engagers and/or mounts 12, 14 can resist over-contraction of the device 10. As another example, the engagers and/or mounts 12, 14 can have an extension configured to contact another extension, engager, or mount when the device 10 is in its maximally contracted state. When in contact, the extension can resist additional contraction of the device 10. The extensions can be, for example, flanges or feet of the engagers and/or mounts 12, 14. To inhibit over-expansion, a linkage can be placed between two or more of the engagers and/or mounts 12, 14 and can be coupled to one or both of them. As another example, the linkage can be placed along the band 16 and attached to the band 16. The linkage can be flexible or rigid. For example, the linkage can be a flexible cable, line, or thread. The linkage can have slack when the device is not fully contracted and can be taut when the when the device 10 is fully contracted. The linkage can thereby resist over-expansion. As yet a further example, the bands 16 can function as stops. For example, the bands 16 can be shape set to have maximum expanded and contracted configurations. In this way, the bands 16 can prevent over-contraction and over-expansion, for example, by becoming taught after unfolding, folding, stretching, compressing, or any combination thereof. The bands 16 can thereby delimit their own expansion and contraction. The device 10 can be capable of further contraction but for the one or multiple stops. The device 10 can be capable of further contraction but for the one or multiple stops.

The maximally contracted state can be quantified as a maximum contraction threshold. The maximally expanded state can be quantified as a maximum expansion threshold. These thresholds can be determined by comparing a parameter of the device 10 between two of the following configurations: a maximally contracted configuration, a neutral configuration, a maximally expanded configuration. The parameter can be the size of the bands 16 (e.g., their perimeter), the length, width, or height of the device 10, the area of the openings 24 or 26, the space between adjacent engagers and mounts 12, 14, or any combination thereof. The contraction and expansion thresholds can be a ratio of any of these values. For example, the parameter of the device 10 in a maximally contracted state can be about 5% to about 500% smaller than the parameter of the device 10 in the neutral or maximally expanded state, including every 10% increment within this range (e.g., 25%, 50%, 75%, or 100%). As another example, the parameter of the device 10 in a maximally expanded state can be about 5% to about 500% larger than the parameter of the device 10 in the neutral or maximally contracted state, including every 10% increment within this range (e.g., 25%, 50%, 75%, or 100%).

The device 10 can have 2 to 20 or more engagers 12, including every 1 engager increment within this range. The device 10 can have 2 to 20 or more mounts 14, including every 1 mount increment within this range. The number of engagers 12 can be the same or different than the number of mounts 14. The device 10 can have 1 to 10 or more bands 16, including every 1 band increment within this range.

The device 10 can have a height. The height can be measured along an axis perpendicular to the surface of the medium 8. The height can be, for example, about 2 mm to about 15 mm, including every 1 mm increment within this range (e.g., 2 mm, 4 mm, 6 mm, 10 mm). With such heights, the device 10 can have a low profile so that it can, for example, be worn under clothing. The low profile of the device 10 can limit the interference that the device 10 causes to a user's movement when wearing the device 10.

The device 10 can include a tissue dressing (e.g., a wound dressing). As another example, the device 10 can be attached to or integrated with a wound dressing.

The device 10 can include a negative pressure wound therapy device. As another example, the device 10 can be attached to or integrated with a negative pressure wound therapy device. The device 10 and/or the negative pressure wound therapy device can be connected to a negative pressure source and/or can have a pump that can generate a negative pressure.

The device 10 can have electronics including one or more sensors, processors, power sources and transceivers. For example, the device 10 can have a temperature sensor that can monitor the temperature of the target tissue 6. The device 10 can have a chemical sensor that can recognize one or more fluid components to be monitored in wound exudate that weeps from the target tissue 6. The sensors can be attached to or integrated with one or more engagers 12, one or more mounts 14, one or more bands 16, the cover 30, the flap 32, the skirt 40, the wound dressing, the negative pressure wound therapy system 33, or any combination thereof.

The device 10 can have a wound exudate reservoir that can collect wound exudate as it weeps from the target tissue 6.

The device 10 can have one or multiple solutions such as natural and/or artificial tissue treatment agents, medicine (e.g., anti-inflammatory drugs, local anesthetics), antibiotics, nutrients, creams, lotions, moisturizers, or any combination thereof. For example, a cover and/or a tissue dressing can have any combination of solutions for direct application to the body, for indirect application to the body, or both. As another example, the device 10 can have one or multiple solution reservoirs, where each reservoir can have any combination of one or multiple solutions. The device 10 can spray the solutions onto tissue, can directly apply the solutions to tissue via a tissue cover (also referred to throughout as a wound cover), or any combination thereof. The solutions can be, for example, a liquid or a gel. The device 10 can apply the one or multiple solutions to the skin.

The solutions be released into tissue, onto tissue (e.g., a topical application), or both. For example, the solutions can be released into and/or onto the target tissue 6. The solutions can be released from the device 10 all at once or time-released over, for example, 1 to 8 hours, 1-24 hours, 1 day to 7 days. The device 10 can release the solutions at a constant rate or at a varied rate over a predetermined time period. The device 10 can release solutions independent of tissue conditions, for example, independent of the target tissue condition, independent of the tissue condition surrounding the target tissue, or both. As another example, the device 10 can release solutions dependent on tissue conditions, for example, dependent on the target tissue condition, dependent on the tissue condition surrounding the target tissue 6, or both. For example, the device 10 can release one or multiple solutions based on one or more conditions near the device 10, including, for example, temperature conditions, chemical conditions, wound exudate flow conditions, target tissue wetness or dryness conditions, user pain conditions, or any combination thereof. The device 10 can determine or sense these conditions, for example, using one or more sensors (e.g., using a temperature sensor, a chemical sensor, a wetness sensor, a flow sensor, or any combination thereof). The temperature conditions can be a tissue temperature, an environmental temperature, or both. For example, the temperature condition be the temperature of the target tissue, of tissue surrounding the target tissue, of the air temperature outside the device 10, of the air inside the device 10 (e.g., where there is a wound covering), or any combination thereof. The chemical conditions can be, for example, the chemical composition of tissue exudate (e.g., wound exudate). For user pain conditions, the device 10 can have or be associated with a controller (e.g., wired or wirelessly) in which the user can input their level of pain, for example, on a scale from 1-10, where 1 is the lowest pain level and 10 is the highest pain level, such that solutions are releasable from the device 10 based on the input from the user. For example, the device 10 can release more solution for higher pain levels than lower pain levels, or vice versa.

The device 10 can have one or multiple patches configured to deliver one or multiple solutions onto tissue, into tissue, or both. For example, the device 10 can have one or multiple medicated patches. The device 10 can have one or multiple iontophoreas patches and/or one or multiple electroporation patches to help push medication into tissue. The patches can be removably attachable to the device 10. The patches can be disposable and can be replaced or changed with and without the device 10 being attached to a user's body. The patches can be separate from the tissue dressing. The patches can be the tissue dressing. The patches can be integrated with or attached to the tissue dressing.

The device 10 can be a single-use device or a multiple-use device. The device 10 can be disposable. The device 10 can be sterilizable.

A user can wear the device 10 while awake, while asleep, or both.

The device 10 can include a suture passer or can be sutureless device.

The device 10 can include a tissue cutter.

The device 10 can function as a tissue spreader, a tissue closer, a tissue mover, or any combination thereof. The device 10 can function as a wound opener, a wound closer, a tissue mover, or any combination thereof. The device 10 can open wounds, close wounds, move tissue, or any combination thereof. The device 10 can be a tissue mover. The device 10 can be a solution applier, where the device 10 can apply solution to the target tissue. The device 10 can be a target tissue protector, where the device 10 can be a physical barrier to protect the target tissue 6.

FIGS. 7A-7D illustrate that the device 10 can conform to the shape of the tissue 8. For example, the device 10 can conform to the shape of the surface of the body, for example, the surface of a limb, torso (e.g., back, abdomen, chest), neck, head (e.g., forehead, cheeks), or any combination thereof. The device 10 can conform to a flat surface. The device 10 can be positionable on a flat surface. The device 10 can conform to a curved surface. The device 10 can be positionable on a curved surface. The device 10 can flex for example, along any axis away from a flat planar surface. The device 10 can conform to a flat surface, convex surface, a concave surface, or any combination thereof. The device neutral configuration can be a flat configuration, a curved configuration, or both. The device 10 can be designed for a flat surface. The device 10 can be designed for a curved surface. A device 10 designed for a flat surface can be positionable on a flat surface, a curved surface, or both. A device 10 designed for a flat surface can conform to a flat surface or to a surface having any curvature (e.g., convex and/or concave). A device 10 designed for a curved surface can be positionable on a flat surface, a curved, surface, or both. A device 10 designed for a curved surface can conform to a flat surface or to a surface having any curvature (e.g., convex and/or concave). When the device 10 is positioned on the tissue 8, the bands 16 can contact or not contact the tissue 8.

FIGS. 7A and 7B illustrate that the device 10 can be attached to a non-flat tissue surface.

FIGS. 7C and 7C illustrate that the device 10 can be attached a flat tissue surface.

FIG. 8 illustrates that the device 10 can have one or multiple tabs 42. The tabs 42 can be pull tabs. The tabs 42 can be the attachment tool. The device 10 can have, for example, 1 to 10 or more tabs 42, including every 1 tab increment within this range (e.g., 2 tabs, 4 tabs 10 tabs). The device 10 can have the same number of tabs as engagers 12. The device 10 can have 1 tab 42 per 2 to 10 engagers 12, including every 1 tab increment within this range (e.g., one tab 12 per two engagers 12, one tab 12 per three engagers 12, one tab 12 per four engagers 12), or any combination thereof. The tabs 44 can cover adhesive on the engagers 12. Once removed (e.g., pulled) from the engagers, for example, as shown by removal (e.g., pull) arrows 44, the adhesive can be exposed. Once the adhesive is exposed, the device 10 can be removably attached to the tissue 8 via the engagers 12. The tabs 42 can be paper, plastic, or a composite material. For example, the tabs 42 can be cardboard pull tabs. The tabs 42 can be removed together or in any sequence where the device 10 has multiple tabs 42 (e.g., the device 10 in FIG. 8). The device 10 can be expanded or contracted (e.g., where the arrows 44 are oriented in the opposite direction than that shown in FIG. 8) entirely or at least partly from the removal of the tabs 42 from the device 10. As another example, the tabs 42 can be removed from the device 10 independently from the expansion or contraction of the device 10, which can be done with or without a tool and/or with or without one or two hands.

The device first configuration can be a device expanded configuration, for example, a partially expanded configuration or a maximum expanded configuration. The device first configuration can be a device contracted configuration, for example, a partially expanded configuration or a maximum contracted configuration. The device first configuration can be a device neutral configuration. The device neutral configuration can be a device expanded configuration, for example, a device partially expanded configuration or a device maximum expanded configuration. The device neutral configuration can be a device contracted configuration, for example, a device partially contracted configuration or a device maximum contracted configuration.

The device second configuration can be a device expanded configuration, for example, a partially expanded configuration or a maximum expanded configuration. The device second configuration can be a device contracted configuration, for example, a partially expanded configuration or a maximum contracted configuration. The device second configuration can be a device neutral configuration. The device neutral configuration can be a device expanded configuration, for example, a device partially expanded configuration or a device maximum expanded configuration. The device neutral configuration can be a device contracted configuration, for example, a device partially contracted configuration or a device maximum contracted configuration. The device contracted configuration can be a closed configuration. The device expanded configuration can be an open configuration. The device first configuration can be less open than the device second configuration, or vice versa. The device contracted configuration can be less open than the device expanded configuration such that the tissue access port has a smaller area in the device contracted configuration than in the device expanded configuration.

When the device 10 changes from the device first configuration to the device second configuration, a length of the device can increase from the dimension L₁ to the dimension L₂. When the device changes from the device first configuration to the device second configuration, a length of the device can decrease from the dimension L₁ to the dimension L₂.

When the device 10 changes from the device second configuration to the device first configuration, a length of the device can increase from the dimension L₁ to the dimension L₂. When the device changes from the device second configuration to the device first configuration, a length of the device can decrease from the dimension L₁ to the dimension L₂.

### Method of Use

The device 10 can be expanded and/or contracted with or without a tool. The tool can include an elongate shaft. The tool shaft can have a length greater than a width of the device 10.

The device 10 can be attached to the medium 8 with or without a tool. The tool can include an elongate shaft. The tool shaft can have a length greater than a width of the device 10. The tool can be the tabs 42.

The tool that can expand and/or contract the device 10 can be the same as or different from the tool that can attach the device 10 to the medium 8. For example, the tool can be a contractor, an expander, an applicator, or any combination thereof. The tool can be removably attached to the device 10. For example, the tool can be removably attached to one or more engagers, mounts and/or bands 12, 14, 16 of the device 10. Additionally or alternatively, the device 10 can have a tool connector that the tool can attach to.

The device 10 can have the same or different shape in the contracted and expanded configurations. The device 10 can have an increasable size, a decreasable size, or both. For example, the device 10 can have an increasable outer perimeter, a decreasable outer perimeter, or both. The device 10 can have a maximum size and a minimum size. For example, the device can have larger size when the device is in the device second configuration (e.g., FIG. 1B) than when the device is in the device first configuration (e.g., FIG. 1A).

FIG. 1A illustrates that the device 10 can be in a partially or fully contracted configuration before the device 10 is expanded and then placed on the medium 8. FIG. 1A further illustrates that the device 10 can return or be returned to a partially or fully contracted configuration after the device 10 is attached to the medium 8.

FIG. 1B illustrates that the device 10 can be partially or fully expanded and then placed on the medium 8.

FIGS. 1A and 1B illustrate that before the device 10 is placed on the medium 8, the device 10 can be expanded from a device first configuration having a device first size (e.g., FIG. 1A) to a device second configuration having a device second size larger than the device first size (e.g., FIG. 1B). Once the device 10 is in the device second configuration (e.g., FIG. 1B), the device 10 can be contracted to a size smaller than the device second size. For example, the device 10 can be contracted to a size smaller than the second size, can be returned to the device first configuration having the first size (e.g., FIG. 1A), or can be contracted to a size between the first size and the second size. As another example, FIGS. 1A and 1B illustrate that before the device 10 is placed on the medium 8, the device 10 can be contracted from the device second configuration having a device second size (e.g., FIG. 1B) to the device first configuration having the device first size (e.g., FIG. 1A). Once the device 10 is in the device first configuration (e.g., FIG. 1A), the device 10 can be expanded to a size larger than the device first size. For example, the device 10 can be expanded to a size larger than the first size, can be returned to the device second configuration having the second size (e.g., FIG. 1B), or can be expanded to a size between the first size and the second size. The device first configuration can be the initial configuration of the device 10. The device first configuration can be the neutral configuration of the device 10. The device second configuration can be the initial configuration of the device 10. The device second configuration can be the neutral configuration of the device 10.

For example, one hand, two hands, a tool (e.g., an applicator, tabs 42), or any combination thereof (also referred to collectively as an expander) can expand the device 10 from the closed state in FIG. 1A to the open state in FIG. 1B. The expander can expand the device 10 before the device 10 is attached to the medium 8. Once the device 10 is expanded, the same or a different expander can be used to attach the device 10 to the medium 8 while the device 10 is in the expanded state. Once the expander is detached or removed from the device 10, the device 10 can return (e.g., passively) or can be returned (e.g., actively) to its closed position (e.g., its shape set closed position) to close the target tissue 6. The device 10 can return to a more closed configuration (e.g., the device first configuration) via force from only the bands 16. Such closure is also referred to as passive closure. As another example, the device can be returned to a more closed configuration via force from the bands 16 and an external force (e.g., from a closer). Such assisted closure is also referred to as active closure. As yet another example, the device 10 can return or be returned to a closed configuration while the expander is attached to the device 10. As still yet another example, the device 10 can be locked in an expanded state (e.g., a partially expanded state or a fully expanded state) after the device 10 is expanded (e.g., with the tool) such that the device 10 can remain in the expanded state when the expander is removed. The device 10 can have a closing trigger that can be activated after the device 10 is attached to the medium 8. When the closing trigger is activated, the device 10 can return or returned to its shape set closed configuration to close the wound. The force of the band 16 closing the device 10 can close the target tissue 6 and can maintain the device 10 in a closed state. The force that the device 10 imposes on the medium 8 can be greater than the force that would otherwise keep the target tissue 6 open.

The device 10 can be biased to contract from the device configuration shown in FIG. 1B to the device configuration shown in FIG. 1A. For example, FIG. 1A illustrates the device 10 in a relaxed state (also referred to as a neutral state) and FIG. 1B illustrates the device in a tensioned state. When the device 10 is in the tensioned state, the device 10 can be biased to return to the relaxed state. As another example, FIG. 1A illustrates the device 10 in a tensioned (e.g., contracted) state and FIG. 1B illustrates the device 10 in a more tensioned state relative to the tensioned state illustrated in FIG. 1A. When the device 10 is in the more tensioned state, the device 10 can be biased to return to any lesser tensioned state, for example, to the lesser tensioned state shown in FIG. 1A.

As another example, one hand, two hands, a tool (e.g., an applicator, tabs 42), or any combination thereof (also referred to collectively as a compressor) can contract the device 10 from the open state in FIG. 1B to the closed state in FIG. 1A. The compressor can contract the device 10 before the device 10 is attached to the medium 8. Once the device 10 is contracted, the same or a different compressor can be used to attach the device 10 to the medium 8 while the device 10 is in the contracted state. Once the compressor is detached or removed from the device 10, the device 10 can return (e.g., passively) or can be returned (e.g., actively) to its open position (e.g., its shape set closed position) to open the target tissue 6 or to push tissue toward the target tissue 6. The device 10 can return to an open configuration (e.g., the device second configuration) via force from only the bands 16. Such opening is also referred to as passive opening. As another example, the device can be returned to a more open configuration via force from the bands 16 and an external force (e.g., from a closer). Such assisted closure is also referred to as active closure. As yet another example, the device 10 can return or be returned to an open configuration while the compressor is attached to the device 10. As still yet another example, the device 10 can be locked in a contracted state (e.g., a partially contracted state or a fully contracted state) after the device 10 is contracted (e.g., with the tool) such that the device 10 can remain in the contracted state when the compressor is removed. The device 10 can have an opening trigger that can be activated after the device 10 is attached to the medium 8. When the opening trigger is activated, the device 10 can return or returned to its shape set open configuration to open to push tissue toward the target tissue 6. The force of the band 16 opening the device 10 can open the target tissue 6, can push tissue toward the target tissue 6, can maintain the device 10 in an open state, or any combination thereof. The force that the device 10 imposes on the medium 8 can be greater than the force that would otherwise keep the target tissue 6 closed.

The device 10 can be biased to expand from the device configuration shown in FIG. 1A to the device configuration shown in FIG. 1B. For example, FIG. 1B illustrates the device 10 in a relaxed state (also referred to as a neutral state) and FIG. 1A illustrates the device in a tensioned (e.g., contracted) state. When the device 10 is in the tensioned state, the device 10 can be biased to return to the relaxed state. As another example, FIG. 1B illustrates the device 10 in a tensioned (e.g., contracted) state and FIG. 1A illustrates the device 10 in a more tensioned (e.g., more contracted) state relative to the tensioned state illustrated in FIG. 1B. When the device 10 is in the more tensioned state, the device 10 can be biased to return to any lesser tensioned state, for example, to the lesser tensioned state shown in FIG. 1B.

FIGS. 1A and 1B further illustrate that the target tissue 6 can be a port wound. The port target tissue 6 can have a port size of about 1 cm to about 30 cm or more, including every lcm increment within this range. This dimension can be the diameter of the port wound.

FIGS. 1A and 1B further illustrate that the device 10 can close around a device center axis A₁. The device center axis A₁ can be a center axis of the device 10 and can pass through the center of the openings 24 and/or 26.

FIGS. 1A and 1B further illustrate that the device 10 can increase in length and/or decrease in length along the device longitudinal axis with or without expansion and/or contraction along a transvers axis perpendicular to the device longitudinal axis.

FIGS. 1A and 1B further illustrate that the device 10 can radially expand and contract. The expansion and contraction can be symmetrical toward and away from the device transverse axis.

FIGS. 1A and 1B further illustrate that the device 10 can be the same distance from an edge of the target tissue 6 in the contracted and expanded configurations before the target tissue 6 begins to heal.

FIGS. 2A and 2B illustrate that the device 10 can expand and contract with the tissue cover 30.

FIGS. 3A and 3B illustrate that the device 10 can expand and contract with the tissue cover 30 and the flap 32.

FIGS. 4A and 4B illustrate that the device 10 can expand and contract with the tissue cover 30, the flap 32 and the negative pressure wound therapy system 33.

FIGS. 5A and 5B illustrate that the device 10 can asymmetrically expand and/or contract. For example, the device 10 can expand and contract along the device first transverse axis A₂ but not along the device second transverse axis A₃. As another example, the device 10 can expand and contract along the first and second axes A₂, A₃, but can expand and contract along the second axis A₃ less than along the first axis A₂. As yet another example, the device 10 can expand and contract along the first and second axes A₂, A₃, but can expand and contract along the second axis A₃ more than along the first axis A₂.

FIGS. 6A and 6B illustrate that the device 10 can tension and/or compress a medium 8 having stiches.

The device 10 can close the target tissue 6 with or without extending across the target tissue 6 after being attached to the medium 8 in an expanded configuration (e.g., maximally expanded state). During attachment of the device 10 to the medium and/or during contraction of the device 10 while attached to the medium, the openings 24 and 26 can remain unobstructed. However, FIG. 6A illustrates that tensioners 28 can extend across the openings 24 and 26 before, during, and after contraction.

FIGS. 7A and 7B illustrate that the device 10 can be attached to a non-flat medium 8. For example, the device 10 can be attached to a curved medium 8. The medium 8 can have a convex curve, a concave curve, or both. The device 10 can be attached to a medium have a radius of curvature of any body part. For example, the device 10 can be attached to the user's head, face (e.g., forehead, cheek, nose, chin), neck, back, chest, arms, wrists, hands, fingers, legs, ankles, feet, and/or toes. The device 10 can be attached to a single body part. The device 10 can be attached to multiple body parts simultaneously, for example, a user's cheek and chin.

FIGS. 7C and 7D illustrate that the device 10 can be attached to a flat medium 8.

FIG. 8 illustrates that the device 10 can be attached to and removed from the medium 8 with tabs 42.

The device 10 can be controllably adjusted (e.g., expanded and/or contracted). For example, the state of the device 10 can be incrementally changed, for example, as measured relative to a maximum expansion and/or contraction percentage. For example, the device 10 can be expanded in about 5% to about 25% increments, including every 5% increment within this range (e.g., 0% expanded, 25% expanded, 50% expanded, 75% expanded, 100% expanded). Such incremental adjustments can be done over a duration of 10 seconds or more, 1 day or more, or 1 week or more.

The device 10 can tension tissue adjacent a skin graft. The device 10 can compress tissue adjacent a skin graft. The device 10 can tension and compress tissue adjacent a skin graft. This can desirably provide the skin graft with a more stable growth surface.

The device 10 can tension tissue having stitches. The device 10 can compress tissue having stitches. The device 10 can tension and compress tissue having stitches. This can desirably help stabilize the tissue near the stiches, which can in turn reduce the strain on both the stiches and surrounding skin. With less strain, a smaller suture and/or fewer passes of the suture through the tissue can be used.

The device 10 can automatically return to a neutral position when released from a non-neutral position. The device 10 can be manually returned to a neutral position when released from a non-neutral position (e.g., a shape set state, an expanded state, a contracted state, or any combination thereof).

Large, medium, and/or small devices 10 can be successively placed on the medium 8 to adjust for the new tissue growth during the healing process.

The device 10 can release antibiotics, nutrients, or both into the target tissue 6. For example, a cover and/or a wound dressing having antibiotics and/or nutrients can release them into the target tissue 6 over time or all at once. As another example, the cover and/or the wound dressing can be or can include one or more patches, for example, iotophoreas patches, electroporation patches, or both.

FIGS. 9A-9E illustrate that the device 10 can be placed partially or entirely around a target tissue 6, for example, to close a wound, to close a lesion, to reduce tissue tension at the target tissue 6 (e.g., a pustule), or any combination thereof. When or after the device 10 is placed around a target tissue 6, the device 10 can be decreased in size and/or can decrease in size to close the target tissue 6, for example, by moving from a larger configuration (e.g., the device second configuration in FIG. 9A) to a smaller configuration (e.g., the device first configuration in FIG. 9B). As another example, when or after the device 10 is placed around the target tissue 6, the device 10 can be decreased in size and/or can decrease in size to urge (e.g., push) skin and/or underlying tissue toward the target tissue 6, for example, by moving from a larger configuration (e.g., the device second configuration in FIG. 9A) to a smaller configuration (e.g., the device first configuration in FIG. 9B). The target tissue can be one or multiple pustules (e.g., acne). For example, FIGS. 9A and 9B illustrate that the device 10 can be attached to tissue so that it can entirely surround three pustules. However, the device 10 can be attached to tissue having 1 to 50 or more pustules, including every 1 pustule increment within this range. FIG. 9A illustrates the device 10 in a tensioned state and FIG. 9B illustrates the device in a relaxed state (also referred to as a neutral state). As another example, FIG. 9A illustrates the device 10 in a tensioned state and FIG. 9B illustrates the device in a less tensioned state relative to the state illustrated in FIG. 9A.

FIGS. 9A and 9B further illustrate that the size of the pustules can decrease when the device 10 contracts from the tensioned state in FIG. 9A to the relaxed state or to the lesser tensioned state in FIG. 9B. For example, the target tissue 6 (e.g., pustules) can have a first size 6_{A1} before the device 10 is contracted and can have a second size 6_{A2} smaller than the first size 6_{A1} after the device 10 is contracted. The first and second sizes 6_{A1}, 6_{A2} can be a measurable feature of the target tissue 6, for example, a length, width, height, surface area, skin depth, perimeter (e.g., circumference), or any combination thereof, of the target tissue 6. For example, FIGS. 9A and 9B illustrate that the device 10 can pull (e.g., radially pull) skin and underlying tissue toward the target tissue 6 to decrease stretching of the target tissue 6, to reduce the pressure on or near the target tissue 6, or both. In this way the device 10 can be placed over the target tissue 6 (e.g., lesions or pustules) to compress, push and/or pull healthy skin toward the target tissue 6, thereby de-tensioning the skin over the target tissue 6.

FIGS. 9A and 9B further illustrates that the skin can have wrinkles 46. The wrinkles 46 can be, for example, wrinkles in the stratum corneum layer, stratum lucidum layer, stratum granulosum layer, stratum spinosum layer, stratum basale layer, dermis layer of the skin, or any combination thereof. FIGS. 9A and 9B illustrate that when the device 10 is contracted (e.g., from the tensioned state in FIG. 9A) to the relaxed state or to the lesser tensioned state in FIG. 9B, that some of the wrinkles 46 can decrease in size (e.g., flatten), that some of the wrinkles 46 can increase in size (e.g., deepen), that one or more new wrinkles 46 can form in the skin, that one or multiple folds 48 (also referred to as over-wrinkling) can form in the skin, that new skin features 50 can form in the skin, or any combination thereof. The new skin features 50 can be, for example, skin wrinkles, skin folds, or both. For example, FIGS. 9A and 9B illustrate that the wrinkles 46 outside the device 10 (e.g., not in the openings 24, 26) can flatten when the device 10 contracts (e.g., where the device 10 stretches the skin), that the wrinkles 46 near or in the target tissue 6 can deepen (e.g., where the device 10 compresses the skin, for example, in the openings 24 and 26), that folds 48 can form in the in the skin in or near the target tissue 6 (e.g., where the device 10 compresses the skin), that new skin features can form in or near the target tissue 6 (e.g., where the device 10 compresses the skin), or any combination thereof. As an example, the wrinkles 46 outside the device 10 in FIG. 9A are shown stretched and not visible in FIG. 9B. FIGS. 9A and 9B illustrate that the device 10 can tension and de-tension tissue. At the locations where the device 10 tensions or stretches tissue, the wrinkles and/or folds 46, 48 in the skin can stretch, become smaller, shallower, and/or wider, become less visible, disappear altogether, or any combination thereof. Some or all of the wrinkles and folds 46, 48 can return in the skin or otherwise be visible again when the tension force applied to the skin via the device 10 is released or lessened. For example, wrinkles that were stretched outside of the device 10 when the device 10 was in a contracted configuration (e.g., the contracted configuration in FIG. 9B) can de-stretch and become visible again when the tension force is released or lessened, for example, by removing the device 10 from the skin or by changing the device 10 to a less contracted configuration (e.g., by changing the device 10 from the contracted configuration in FIG. 9B to the non-contracted configuration in FIG. 9A). At the locations where the device 10 de-tensions or compresses tissue, the wrinkles and/or folds 46, 48 in the skin can bunch up, become larger, deeper, and/or narrower, become more visible, new wrinkles and folds can form, or any combination thereof. Some or all of the wrinkles and folds 46, 48 can return in the skin or otherwise be visible again when the compression force applied to the skin via the device 10 is released or lessened. For example, wrinkles that were inside the device 10 but in a state of tension (e.g., because the wrinkles were part of a skin fold, for example, at a crown or apex of a skin fold) when the device 10 was in a contracted configuration (e.g., the contracted configuration in FIG. 9B) can de-stretch and become visible again when the compression force is released or lessened, for example, by removing the device 10 from the skin or by changing the device 10 to a less contracted configuration (e.g., by changing the device 10 from the contracted configuration in FIG. 9B to the non-contracted configuration in FIG. 9A). FIGS. 9A and 9B thereby illustrate the skin inside the device 10 (e.g., inside the openings 24, 26) getting compressed (also referred to as squished together) and skin outside of the device 10 (e.g., outside of the openings 24, 26) getting stretched.

FIGS. 9A and 9B′ illustrate that the size of the pustules can remain the same when the device 10 contracts from the tensioned state in FIG. 9A to the relaxed state or to the lesser tensioned state in FIG. 9B′. For example, FIGS. 9A and 9B illustrate that the target tissue 6 (e.g., pustules) can have the first size 6_{A1} in both the pre-contracted and post-contracted configurations.

FIGS. 9C and 9D illustrate one of the pustules inside of the device 10. FIG. 9C illustrates that the target tissue 6 (e.g., skin and underlying tissue near the pustule) can be less stretched in FIG. 9D than in FIG. 9C, shown for example, by the sizes of wrinkles 46 being larger, deeper, and/or narrower in FIG. 9D than in FIG. 9C, by the wrinkles 46 being more visible in FIG. 9D than in FIG. 9C, by the appearance of new wrinkles and/or folds in FIG. 9D not present in FIG. 9C, or any combination thereof. FIGS. 9C and 9D further illustrate that the target tissue 6 can extend a target tissue height 6_{H} away from a surface of surrounding skin, for example, due to inflammation in the target tissue 6. The surrounding skin can be part of the target tissue 6 or can be different from (e.g., adjacent to) the target tissue 6. The target tissue height 6_{H} can be the same or different when the device 10 is in a non-contracted state (e.g., before the device 10 is contracted) than when the device is in a contracted state (e.g., after the device 10 is contracted). By tensioning and/or de-tensioning the target tissue 6, the device 10 can increase, maintain, or decrease the target tissue height 6_{H} when the device is changed from a first configuration to a second configuration or vice versa, for example, from a device non-contracted configuration to a device contracted configuration or vice versa. Compression of the tissue caused by the device 10 in the target area can decrease the target tissue height 6_{H}, increase the target tissue height 6_{H}, or both. For example, FIGS. 9C and 9D further illustrate that pre-device contraction, the target tissue 6 can have a target tissue first height 6_{H1}, and that post-device contraction, the target tissue 6 can have a target tissue second height 6_{H2} the same as, greater than, or less than the target tissue first height 6_{H1}, which can depend on the reference point chosen to establish the base of the of the measured height. For example, the base can be chosen a fixed radial distance away from a center of the target tissue 6 or can be chosen to be measured from where the skin is flat relative to the target tissue 6.

FIG. 9E illustrates a solution 45 (e.g., therapeutic ointment) applied on top of the target tissue 6 (e.g., pustule, lesion). The de-tensioning of the skin can enhance the local absorption of the solution 45 at and/or near the target tissue 6. As another example, no solution 45 or medication might be used, and the de-tensioning alone can provide a beneficial healing effect, for example, by reducing scarring, by increasing the healing rate, or both.

FIGS. 9A-9E further illustrate that the device 10 can be closer to the target tissue 6 in the device treatment configuration (e.g., the device contracted configuration, the device first configuration) than in the device pre-treatment configuration (e.g., the device expanded configuration, the device second configuration). For example, FIGS. 9A-9E illustrate that one or more of the engagers 12, one or more of the mounts 14, the bands 16, or any combination thereof can be closer to the target tissue 6 in the device treatment configuration (e.g., the device contracted configuration, the device first configuration) than in the device pre-treatment configuration (e.g., the device expanded configuration, the device second configuration).

FIGS. 10A-10E illustrate that the device 10 can be placed next to the target tissue 6, for example, to close a wound, to close a lesion, to reduce tissue tension at the target tissue 6 (e.g., a pustule), or any combination thereof. When the device 10 is placed next to a target tissue 6, the device 10 can be increased in size and/or can increase in size to push tissue toward the target tissue 6 to close the wound or relieve pressure on the target tissue 6, for example, by moving from a smaller configuration (e.g., the device first configuration in FIG. 10A) to a larger configuration (e.g., the device second configuration in FIG. 10B). As another example, when or after the device 10 is placed next to the target tissue 6, the device 10 can be increased in size and/or can increase in size to urge (e.g., push) skin and/or underlying tissue toward the target tissue 6, for example, by moving from a smaller configuration (e.g., the device first configuration in FIG. 10A) to a larger configuration (e.g., the device second configuration in FIG. 10B). The target tissue can be one or multiple pustules (e.g., acne). For example, FIGS. 10A and 10B illustrate that the device 10 can be attached to tissue so that it can be next to or partially surround three pustules. However, the device 10 can be attached next to tissue having 1 to 50 or more pustules, including every 1 pustule increment within this range. FIG. 10A illustrates the device 10 in a relaxed state and FIG. 10B illustrates the device in a relaxed state (also referred to as a neutral state). As another example, FIG. 10A illustrates the device 10 in a tensioned state and FIG. 10B illustrates the device in a less tensioned state relative to the state illustrated in FIG. 10A.

FIGS. 10A and 10B further illustrate that the size of the pustules can decrease when the device 10 expands from the tensioned state in FIG. 10A to the relaxed state or to the lesser tensioned state in FIG. 10B. For example, the target tissue 6 (e.g., pustules) can have a first size 6_{A1} before the device 10 is expanded and can have a second size 6_{A2} smaller than the first size 6_{A1} after the device 10 is expanded. The first and second sizes 6_{A1}, 6_{A2} can be a measurable feature of the target tissue 6, for example, a length, width, height, surface area, skin depth, perimeter (e.g., circumference), or any combination thereof, of the target tissue 6. For example, FIGS. 10A and 10B illustrate that the device 10 can push (e.g., radially push) skin and underlying tissue toward the target tissue 6 to decrease stretching of the target tissue 6, to reduce the pressure on or near the target tissue 6, or both. In this way the device 10 can be placed near (e.g., but not over) the target tissue 6 (e.g., lesions or pustules) to compress or push healthy skin toward the target tissue 6, thereby de-tensioning the skin over the target tissue 6. FIGS. 10A and 10B further illustrates that the skin can have wrinkles 46. The wrinkles 46 can be, for example, wrinkles in the stratum corneum layer, stratum lucidum layer, stratum granulosum layer, stratum spinosum layer, stratum basale layer, dermis layer of the skin, or any combination thereof. FIGS. 10A and 10B illustrate that when the device 10 is expanded (e.g., from the tensioned state in FIG. 10A) to the relaxed state or to the lesser tensioned state in FIG. 10B, that some of the wrinkles 46 can decrease in size (e.g., flatten), that some of the wrinkles 46 can increase in size (e.g., deepen), that one or more new wrinkles 46 can form in the skin, that one or multiple folds 48 (also referred to as over-wrinkling) can form in the skin, that new skin features 50 can form in the skin, or any combination thereof. The new skin features 50 can be, for example, skin wrinkles, skin folds, or both. For example, FIGS. 10A and 10B illustrate that the wrinkles 46 inside the device 10 (e.g., in the openings 24, 26) can flatten when the device 10 expands (e.g., where the device 10 stretches the skin), that the wrinkles 46 near or in the target tissue 6 can deepen (e.g., where the device 10 compresses the skin), that folds 48 can form in the in the skin in or near the target tissue 6 (e.g., where the device 10 compresses the skin), that new skin features can form in or near the target tissue 6 (e.g., where the device 10 compresses the skin), or any combination thereof. As an example, the wrinkles 46 inside the device 10 in FIG. 10A are shown stretched and not visible in FIG. 10B. FIGS. 10A and 10B illustrate that the device 10 can tension and de-tension tissue. At the locations where the device 10 tensions or stretches tissue, the wrinkles and/or folds 46, 48 in the skin can stretch, become smaller, shallower, and/or wider, become less visible, disappear altogether, or any combination thereof. Some or all of the wrinkles and folds 46, 48 can return in the skin or otherwise be visible again when the tension force applied to the skin via the device 10 is released or lessened. For example, wrinkles that were stretched inside of the device 10 when the device 10 was in an expanded configuration (e.g., the expanded configuration in FIG. 10B) can de-stretch and become visible again when the tension force is released or lessened, for example, by removing the device 10 from the skin or by changing the device 10 to a less expanded configuration (e.g., by changing the device 10 from the expanded configuration in FIG. 10B to the non-expanded configuration in FIG. 10A). At the locations where the device 10 de-tensions or compresses tissue, the wrinkles and/or folds 46, 48 in the skin can bunch up, become larger, deeper, and/or narrower, become more visible, new wrinkles and folds can form, or any combination thereof. Some or all of the wrinkles and folds 46, 48 can return in the skin or otherwise be visible again when the compression force applied to the skin via the device 10 is released or lessened. For example, wrinkles that were outside the device 10 but in a state of tension (e.g., because the wrinkles were part of a skin fold, for example, at a crown or apex of a skin fold) when the device 10 was in an expanded configuration (e.g., the expanded configuration in FIG. 10B) can de-stretch and become visible again when the compression force is released or lessened, for example, by removing the device 10 from the skin or by changing the device 10 to a less expanded configuration (e.g., by changing the device 10 from the expanded configuration in FIG. 10B to the non-expanded configuration in FIG. 10A). FIGS. 10A and 10B thereby illustrate the skin outside the device 10 (e.g., outside the openings 24, 26) getting compressed (also referred to as squished together) and skin inside of the device 10 (e.g., inside of the openings 24, 26) getting stretched.

FIGS. 10A and 10B′ illustrate that the size of the pustules can remain the same when the device 10 expands from the tensioned state in FIG. 10A to the relaxed state or to the lesser tensioned state in FIG. 10B′. For example, FIGS. 10A and 10B illustrate that the target tissue 6 (e.g., pustules) can have the first size 6_{A1} in both the pre-expanded and post-expanded configurations.

FIGS. 10C and 10D illustrate one of the pustules outside of the device 10. FIG. 9C illustrates that the target tissue 6 (e.g., skin and underlying tissue near the pustule) can be less stretched in FIG. 10D than in FIG. 10C, shown for example, by the sizes of wrinkles 46 being larger, deeper, and/or narrower in FIG. 10D than in FIG. 10C, by the wrinkles 46 being more visible in FIG. 10D than in FIG. 10C, by the appearance of new wrinkles and/or folds in FIG. 10D not present in FIG. 10C, or any combination thereof.

FIG. 10E illustrates a solution 45 (e.g., therapeutic ointment) applied on top of the target tissue 6 (e.g., pustule, lesion). The de-tensioning of the skin can enhance the local absorption of the solution 45 at and/or near the target tissue 6. As another example, no solution 45 or medication might be used, and the de-tensioning alone can provide a beneficial healing effect, for example, by reducing scarring, by increasing the healing rate, or both.

The device 10 can treat lesions or pustules such as acne on the trunk of the body (e.g., back acne). The device 10 can treat lesions or pustules such as acne on a person's face. The device 10 can treat lesions or pustules by pushing and/or pulling tissue toward the lesions or pustules to de-stretch or de-tension the skin above the lesion or pustule. Decreasing the amount the skin is stretched and tensioned above and/or near the lesions or pustules can advantageously decrease or inhibit scarring and can increase the absorption of topical medical solution (e.g., solution 45).

FIGS. 10A-10E further illustrate that the device 10 can be closer to the target tissue 6 in the device treatment configuration (e.g., the device expanded configuration, the device second configuration) than in the device pre-treatment configuration (e.g., the device contracted configuration, the device first configuration). For example, FIGS. 10A-10E illustrate that one or more of the engagers 12, one or more of the mounts 14, the bands 16, or any combination thereof can be closer to the target tissue 6 in the device treatment configuration (e.g., the device contracted configuration, the device first configuration) than in the device pre-treatment configuration (e.g., the device expanded configuration, the device second configuration).

The solution 45 can include one or more natural and/or artificial tissue treatment agents, medicine (e.g., anti-inflammatory drugs, local anesthetics), antibiotics, nutrients, or any combination thereof. The solution 45 can have any of the properties of any of the solutions described, illustrated and/or contemplated elsewhere in this application. The device 10 can apply the solution 45 to the target tissue 6, for example, every 1 to 24 hours, including every 1 hour increment within this range.

As another example, the device 10 can have no neutral configuration such that the device 10 is in equilibrium and unbiased in any device configuration. In such variations, the device 10 can be manually expanded and/or contracted to achieve the functions and results described in this application with respect to the medium 8 and the target tissue 6.

As yet another example, the device 10—with or without an internal spring—can be manually expanded and/or contracted to achieve the functions and results described in this application with respect to the medium 8 and the target tissue 6. For example, the bands 16 can be biased or unbiased such that the bands 16 can be springs or non-springs. Where the bands 16 are not springs, the device 10 can be manually manipulated with a tool (e.g., an applicator, tabs 42), one hand, two hands, or any combination thereof to increase and/or decrease the size of the device to correspondingly move skin and/or underlying tissue and affect the target tissue 6 as desired (e.g., open a wound, close a wound, decrease pressure on or near a pustule, reduce the tension on or near a pustule, reduce the amount skin above or near a pustule is stretched).

The device 10 can be worn during a treatment period. The treatment period can range, for example, from 5 minutes to 15 minutes, from 5 minutes to 60 minutes, from 1 hour to 24 hours, from 1 hour to 2 or more days, from 1 day to 7 or more days, from 1 day to 30 or more days, less than 90 days, less than 30 days, less than 7 days, less than 1 day, including every 1 minute, 1 hour and 1 day increment within these ranges. The user can wear the device while awake, while asleep, or both.

Although the figures (e.g., FIGS. 9A-10E) show the target tissue 6 being raised relative to surrounding tissue, for example, due to local inflammation, the target tissue 6 can be raised, non-raised (e.g., flat, depressed), or both. As another example, in tissue locations where the device 10 compresses tissue, this can cause the tissue to bunch together. As yet another example, in tissue locations where the device 10 tensions tissue, this can cause the tissue to stretch, flatten out, or both.

The devices 10 can be removably engaged to a user with or without penetrating the user's skin.

U.S. Pat. Nos. 7,455,681; 7,799,042; 8,313,508; 8,439,945; 8,592,640; and 9,179,914 are herein incorporated by reference in their entirety for all purposes. The disclosures in these patents and the present disclosure can be combined and modified with one another in any combination. For example, the device 10 can have any of the features of the devices in these patents. As another example, the methods disclosed herein can include any of the steps and/or tools for carrying on the methods in these patents.

The specific variations described herein are offered by way of example only. The above-described variations, configurations, features, elements, methods and variations of these aspects can be combined and modified with each other in any combination. Any elements described herein as singular can be pluralized (i.e., anything described as “one” can be more than one). Any species element of a genus element can have the characteristics or elements of any other species element of that genus.

We claim:

1. A tissue care device having a device longitudinal axis, comprising:
tissue engagers;
mounts coupled to the tissue engagers; and
a contractible band coupled to the mounts.

2. The tissue care device of claim 1, wherein the contractible band is expandable.

3. The tissue care device of claim 1, wherein the device longitudinal axis is a closed loop.

4. The tissue care device of claim 1, wherein the contractible band defines a device opening.

5. The tissue care device of claim 4, wherein the device opening is configured to provide access to a target tissue.

6. The tissue care device of claim 1, wherein the device is attachable to tissue.

7. The tissue care device of claim 1, wherein the device has a device first configuration and a device second configuration larger than the device first configuration.

8. The tissue care device of claim 7, wherein the device is expandable from the device first configuration to the device second configuration.

9. The tissue care device of claim 8, wherein the device is contractible from the device second configuration to the device first configuration.

10. The tissue care device of claim 9, wherein the device is contractible from the device second configuration to a device third configuration smaller than the device first configuration.

11. A tissue care device having a device longitudinal axis, comprising:
tissue engagers; and
a contractible band coupled to the tissue engagers.

12. The tissue care device of claim 11, wherein the device has a device first configuration and a device second configuration larger than the device first configuration.

13. The tissue care device of claim 12, wherein the contractible band is in a neutral configuration when the device is in the device first configuration and in a non-neutral configuration when the device is in the device second configuration.

14. The tissue care device of claim 12, wherein the contractible band comprises a shape memory alloy, and wherein the contractible band is in a shape set configuration when the device is in the device first configuration and in a strained configuration away from the shape set configuration when the device is in the device second configuration.

15. The tissue care device of claim 11, wherein when the device is attached to tissue, the device is configured to automatically close a target tissue by contracting.

16. The tissue care device of claim 11, wherein the contractible band is a toroid.

17. A method for deploying a tissue care device having a device longitudinal axis, the method comprising:
expanding the tissue care device of claim 11;
attaching the device to tissue having a target tissue; and
contracting the device to partially or entirely close the target tissue.

18. The method of claim 17, wherein expanding comprises expanding the device with or without an expander.

19. The method of claim 17, wherein expanding comprises radially expanding the device.

20. The method of claim 17, wherein expanding comprises the contractible band changing shape.
